(19) United States
(12) Patent Application Publication
(10) Pub. No.: US 2025/0222499 A1
(43) Pub. Date: Jul. 10, 2025

(54) TISSUE DIGESTER DEVICES, SYSTEMS AND METHODS OF USE

(71) Applicant: Philip Jonathon MASON, Sandys (BM)

(72) Inventor: Philip Jonathon MASON, Sandys (BM)

(21) Appl. No.: 18/839,271

(22) PCT Filed: Feb. 18, 2022
(86) PCT No.: PCT/IB2022/051485

§ 371 Date: Aug. 16, 2024

(51) Int. Cl. B09B 3/70; B01J 19/28; B09B 101/65

(52) U.S. Cl. CPC B09B 3/70; B01J 19/28; B09B 2101/65

(57) ABSTRACT

Described are various embodiments of a tissue digester device, a method of introducing a caustic substance to a digestion chamber, a tissue digester system, a method of digesting small tissue bodies, a head encasing device which is operable to mechanically facilitate digestion of brain tissue of a deceased body and a method of facilitating digestion of brain tissue in a head of a deceased body.

### FIELD OF THE DISCLOSURE

The present disclosure relates to the biocremation of tissue remains via alkaline hydrolysis, and, in particular, to tissue digester devices, methods of introducing a caustic substance to a digestion chamber, tissue digester systems, methods of digesting small tissue bodies, head encasing devices which mechanically facilitate digestion of brain tissue and methods of facilitating digestion of brain tissue in a head of a deceased body.

### BACKGROUND

Alkaline hydrolysis, also known as aquamation, biocremation, resomation, flameless cremation or water cremation, is a chemical process for the disposal of human or animal remains. Alkaline hydrolysis is an alternative to the traditional options of land burial or flame incineration (i.e. cremation), which is increasing in popularity due to its comparatively friendly environmental impact. In brief, the alkaline hydrolysis process involves the biochemical hydrolysis of biological tissue in a solution of water and alkaline chemicals (e.g. potassium hydroxide (KOH) and/or sodium hydroxide (NaOH)), with the application of heat and sometimes, pressure. Generally, sex, body mass and weight of the deceased determine the concentration and volume of the digestive fluid.

In the disposal of human remains, the cadaver is typically singularly enclosed within a wire cage and loaded into a digestion chamber of an alkaline hydrolysis system. Water is added to the digestion chamber, to which KOH and/or NaOH in a powdered form is manually added. A digestion cycle is then run, with increased temperature (and sometimes, pressure), for a period of anywhere between three to eighteen hours, depending on equipment design and body mass. Once the digestion cycle is complete, the soft tissue of the deceased is fully dissolved or fluidized as a sterile effluent of salts and amino acids. This effluent is safe for discharge to the environment through a sewerage system or otherwise, can be dispersed as liquid fertilizer. The sterile bones, teeth and remaining mineral content remain at the end of the alkaline hydrolysis process and are manually removed from the wire cage. The undigested residue is typically crushed into a powder for return to an authorized agent of the deceased or otherwise, can be dispersed as a soil additive.

In the case of animals, typically household pets, these can be processed in groups separated individually by physical partitions within the wire cage. Having to adjust the size of the partitions for each pet can be time-consuming and partitions may not be arranged in the most efficient use of space within the wire cage. Since the undigested remains are typically much smaller than human remains, comingling thereof by carriage in the digestive fluid is considered unavoidable. At the end of the digestion process, the remains within each partition are collected (also often time-consuming), pulverized into ash and returned to the owners.

As alkaline hydrolysis for the disposition of human and animal remains is legalised in more states, the industry is working to improving efficiencies of alkaline hydrolysis systems. Whilst some improvements relate to the digestion of less accessible tissues (e.g. brain tissue protected by the cranium), others relate to improving the digestion of multiple smaller animal bodies in a single digestive cycle.

For example, U.S. Pat. No. 8,931,147 entitled “DISPOSAL OF HUMAN REMAINS” and issued Jan. 14, 2015 to Sullivan describes a coffin for use in the alkaline hydrolysis process and methods for disposing of a cadaver. The coffin is a dissolvable receptacle for cadavers and specifically dissolves during the alkaline hydrolysis process. In some embodiments, the coffin includes a bone retaining tray, to which a head-retaining means is affixed at one end. The head-retaining means is typically a tunnel formed of stainless-steel perforated mesh, optionally closed at a crown end, whilst open and flaring out at a neck end. The head-retaining means includes an aperture near the neck end, which is aligned with a recirculation jet when in use such that the recirculation jet is aimed at the head to fully decompose it. The head-retaining means is adapted to restrain movement of the head in all directions throughout the alkaline hydrolysis process and may be in the form of a strap. In some embodiments, the unit further includes a non-dissolvable mesh receptacle (typically comprising a bag). The mesh of the non-dissolvable mesh receptacle is fine enough to retain bone residue but may be prone to fragments getting wedged in the mesh itself, rendering it more difficult to clean between uses. Moreover, such system requires that the non-dissolvable mesh receptacle be fixed to the unit, indicating that there can be no additional motion of the cadaver, which in some cases may desirable to facilitate the digestion of the biological material.

Canadian Patent No. 2,798,117 entitled “TISSUE DIGESTION METHOD AND APPARATUS” and issued Apr. 29, 2011 to Wilson and Wilson discloses various methods and systems for tissue digestion. In some embodiments, the tissue digester includes an elongated cylindrical vessel for holding digestive fluid and a perforated basket(s) for holding tissue to be digested within the vessel. The vessel can be tilted sideways, thereby tilting the basket, such that non-digested tissue fragments are collected in a sloped collection region. The sloped collection region is proximate a propeller-driven mixer for continual agitation of the digestive fluid, thereby progressing digestion.

Canadian Patent No. 3,050,588 entitled “ROTATING TISSUE DIGESTER SYSTEM AND METHOD OF USE” and issued Jan. 26, 2021 to Mason and Monette pertains to a tissue digester system which includes a container for housing a digestion chamber having a perforated interior vessel, one or more heating elements, a motor engaged to create rotational movement of the interior vessel, a control system having a temperature controller and a movement controller. The interior vessel further includes baffles which are attached to and extend from an interior surface of the interior vessel to promote digestion. The control system is operable to rotate the interior vessel and heat the digestion chamber based on user commands.

Canadian Patent Application Publication No. 3,082,241 entitled “REUSABLE ALKALINE HYDROLYSIS CONTAINER AND ALKALINE HYDROLYSIS SYSTEM AND METHOD USING SAME” and published Nov. 28, 2021 to Mason and Monette is generally directed to a digestion container which comprises a non-digestible protective material for preventing digestion of the digestion container. The non-digestible protective material is a copolymer material and is flexible. The flexible digestion container includes one or more re-sealable openings and a plurality of fluid apertures for permitting passage of digestion fluids therethrough. In use, the flexible digestion container (or bag) can be placed within a rotating digestion chamber to retain smaller remains therein.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for tissue digester devices, systems and methods of use that overcome some of the drawbacks of known techniques, or at least, provides a useful alternative thereto. Some aspects of this disclosure provide examples of such devices, systems and methods.

In accordance with a first aspect, there is provided a tissue digester device. The device comprises a rotatable digestion vessel having a body which defines an internal chamber for receiving tissue, a plurality of perforations through the body of the rotatable digestion vessel and a trough formed on the body of the rotatable digestion vessel, the trough extending into the internal chamber and configured to receive a caustic substance when the rotatable digestion vessel is stationary.

In one embodiment, the trough is in the form of a v-shaped notch.

In one embodiment, the trough extends into the internal chamber to form an agitator within the internal chamber.

In one embodiment, the trough extends along a length of the body.

In one embodiment, at least a portion of the trough is impervious to the caustic substance.

In one embodiment, the rotatable digestion vessel comprises a lid which is adapted to provide access to the internal chamber and the trough is formed within the lid.

In one embodiment, the trough comprises at least one wall and the at least one wall in turn, comprises a lid which provides access to a secondary digestion vessel which extends into the internal chamber between the at least one wall and the body.

In one embodiment, the tissue digester device is shaped and dimensioned to be receivable into a digestion chamber such that the caustic substance is insertable into the trough without directly striking fluid held in the digestion chamber.

In one embodiment, the digestion chamber comprises a rotation mechanism and the tissue digester device is connectable to the rotation mechanism.

In one embodiment, rotational movement of the rotatable digestion vessel mixes the caustic substance with fluid in the digestion chamber to form or supplement a digestive fluid.

In one embodiment, the tissue digester device is manufactured of steel.

In accordance with a second aspect, there is provided a method of introducing a caustic substance to a digestion chamber. The method comprises providing the digestion chamber having a rotatable digestion vessel arranged therein, the rotatable digestion vessel having a plurality of perforations and a trough, arranging the rotatable digestion vessel within the digestion chamber such that the trough faces upwards, inserting the caustic substance into the trough whilst stationary and rotating the rotatable digestion vessel such that the caustic substance egresses the trough into the digestion chamber.

In one embodiment, the method comprises inserting fluid in the digestion chamber.

In one embodiment, the rotating of the rotatable digestion vessel mixes the caustic substance with the fluid to form or supplement a digestive fluid.

In one embodiment, the method comprises preloading the rotatable digestion vessel with biological tissue.

In one embodiment, the rotatable digestion vessel comprises a secondary digestion vessel and the method comprises preloading the secondary digestion vessel with biological tissue.

In one embodiment, the fluid comprises water and the caustic substance comprises any one or both of: potassium hydroxide (KOH) and sodium hydroxide (NaOH), or the like.

In one embodiment, the rotatable digestion vessel comprises a tissue digester device as described with reference to the first aspect above.

In accordance with a third aspect, there is provided a tissue digester system. The system comprises a digestion chamber configured to contain a digestive fluid, a rotatable digestion vessel arranged within the digestion chamber, having a substantially perforated body which defines an internal chamber adapted to receive a large tissue body for digestion, and at least one secondary digestion vessel disposable within the internal chamber of the rotatable digestion vessel, having a substantially perforated body which defines a secondary internal chamber adapted to receive a smaller tissue body for digestion. In use, the at least one secondary digestion vessel moves in conjunction with the rotatable digestion vessel such that the large tissue body and the smaller tissue body are simultaneously (at least partially) digested through contact with the digestive fluid.

In one embodiment, the at least one secondary digestion vessel is removably attachable to an inner surface of the rotatable digestion vessel such that it protrudes into the internal chamber. In one embodiment, the at least one secondary digestion vessel is removably attachable to the inner surface with one or more fasteners or the like.

In one embodiment, the rotatable digestion vessel comprises at least one wall and the at least one secondary digestion vessel is formed by the at least one wall protruding into the internal chamber.

In one embodiment, the at least one secondary digestion vessel is freely disposed within the internal chamber of the rotatable digestion vessel, the at least one secondary digestion vessel freely tumbling upon rotation of the rotatable digestion vessel.

In one embodiment, the at least one secondary digestion vessel comprises a floatation pocket which provides floatation to the at least one secondary digestion vessel when in the digestive fluid.

In one embodiment, the at least one secondary digestion vessel comprises a lid through which access to the secondary internal chamber is provided.

In one embodiment, the lid is any one of: a hinged lid, a sliding lid, or the like.

In one embodiment, the at least one secondary digestion vessel is operable as a baffle or an agitator within the internal chamber of the rotatable digestion vessel.

In one embodiment, the at least one secondary digestion vessel is any one of: triangular, circular, square, polygonal or the like, in cross-section.

In one embodiment, the rotatable digestion vessel comprises a trough and the at least one secondary digestion vessel is positioned along a length of an interior wall of the trough.

In one embodiment, the system comprises at least one digestion cannister shaped and dimensioned to receive the smaller tissue body, the at least one digestion cannister receivable into the at least one secondary digestion vessel.

In one embodiment, the at least one digestion cannister comprises a fine mesh.

In one embodiment, the rotatable digestion vessel comprises a tissue digester device as described with reference to the first aspect above.

In accordance with a fourth aspect, there is provided a method of digesting small tissue bodies. The method comprises providing a tissue digester system comprising a digestion chamber configured to contain a digestive fluid, a rotatable digestion vessel arranged within the digestion chamber, having a substantially perforated body which defines an internal chamber, and at least one secondary digestion vessel disposed within the internal chamber of the rotatable digestion vessel, having a substantially perforated body which defines a secondary internal chamber; loading a small tissue body to be digested into the secondary internal chamber of the at least one secondary digestion vessel; adding digestive fluid to the digestion chamber; and rotating the rotatable digestion vessel for a predetermined period to digest the small tissue body through exposure to the digestive fluid in a digestion cycle.

In one embodiment, the tissue digester system further comprises at least one digestion cannister receivable into the secondary internal chamber of the at least one secondary digestion vessel. In one embodiment, the loading of the small tissue body comprises loading the small tissue body into the at least one digestion cannister and inserting the at least one digestion cannister into the secondary internal chamber of the at least one secondary digestion vessel. In one embodiment, the method comprises pausing the digestion cycle of the tissue digester system to remove the at least one digestion cannister from the at least one secondary digestion vessel upon sufficient digestion of the small tissue body.

In one embodiment, the method comprises loading a large tissue body into the internal chamber of the rotatable digestion vessel to be simultaneously digested with the small tissue body.

In one embodiment, the adding of the digestive fluid comprises separately adding to the digestion chamber water and a caustic substance.

In one embodiment, the small tissue body comprises a carcass under one kilogram in body weight.

In one embodiment, the tissue digester system comprises a tissue digester system as described with reference to the third aspect above.

In accordance with a fifth aspect, there is provided a head encasing device which is operable to mechanically facilitate digestion of brain tissue of a deceased body. The head encasing device comprises a body which is shaped and dimensioned to receive and secure therein a head of the deceased body, an aperture formed in the body which is shaped and dimensioned to surround a neck of the deceased body, and an internal projection extending from an internal surface of the body proximate the aperture to engage a craniocervical region of the head, in use to pierce the craniocervical region such that digestive fluid can enter a cranium of the head to digest the brain tissue.

In one embodiment, the internal projection is arranged to pierce the craniocervical region in use such that the digestive fluid can enter the foramen magnum of the head.

In one embodiment, the device comprises an opposing internal projection arranged substantially opposite the internal projection, the internal projection and the opposing internal projection forming a pair of internal projections which engage the craniocervical region at an anterior position and a posterior position, respectively. In one embodiment, the pair of internal projections comprise a pair of spikes.

In one embodiment, the device comprises one or more agitator projections extending from the internal surface of the body.

In one embodiment, the one or more agitator projections are arranged on the internal surface to engage temple regions of the head.

In one embodiment, the one or more agitator projections are configured to fracture one or both pterions of the cranium on lateral sides of the head.

In one embodiment, the body of the device is at least partially perforated such that it is permeable to the digestive fluid.

In one embodiment, the body comprises a two-part body. In one embodiment, the two-part body comprises a hinged connection.

In one embodiment, the body comprises a releasable fastener for securing the body in a closed configuration around the head and the neck.

In one embodiment, the head is a human head and the body is substantially ovoid-shaped.

In one embodiment, the device is manufactured of one or more non-digestible materials such that the head encasing device is reusable for a plurality of digestive cycles.

In one embodiment, any one or both of the body and the internal projection is manufactured of steel.

In accordance with a sixth aspect, there is provided a method of facilitating digestion of brain tissue in a head of a deceased body. The method comprises loading the deceased body into a rotatable digestion vessel of a tissue digester system, fitting a head encasing device to the head of the deceased body such that internal projections disposed within the head encasing device engage a craniocervical region of the deceased body, inserting digestive fluid into the tissue digester system, and rotating the rotatable digestion vessel such that the rotation thereof causes the head encasing device to impact an internal surface of the rotatable digestion vessel, urging the internal projections to pierce the craniocervical region to facilitate entry of the digestive fluid into a cranium of the head for digestion of the brain tissue.

In one embodiment, the piercing of the craniocervical region to facilitate contact between the digestive fluid and the brain tissue reaches the foramen magnum of the cranium.

In one embodiment, the head encasing device comprises one or more agitator projections arranged on an internal surface of the head encasing device to engage temple regions of the head. In use of this embodiment, impact of the head encasing device with the internal surface of the rotatable digestion vessel urges the one or more agitator projections to fracture one or both pterions of the cranium.

In one embodiment, impact of the head encasing device with the internal surface assists in detachment of the head from the deceased body.

In one embodiment, the head encasing device comprises a hinged two-part body and the fitting of the head encasing device to the head comprises opening the hinged two-part body, inserting the head in one part of the hinged two-part body, and closing another part of the hinged two-part body such that the hinged two-part body is secured to the head.

In one embodiment, the head encasing device is a head cage device as described with reference to the fifth aspect above.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a three-dimensional view of a tissue digester device having a trough filled with a caustic substance, with a cutaway view of one end showing the internal chamber and side profile of the trough, in accordance with one embodiment of the present disclosure;

FIG. 2 is a three-dimensional view of the tissue digester device of FIG. 1, housed within a digestion chamber in which a fluid can be retained, in accordance with another embodiment of the present disclosure;

FIG. 3 is a flow diagram of a method of introducing a caustic substance to a digestion chamber, which utilizes a tissue digestor device, in accordance with another embodiment of the present disclosure;

FIG. 4 is a three-dimensional view of a tissue digester system having three independently accessible secondary digestion vessels formed within the rotatable digestion vessel at the trough, each secondary digestion vessel being independently accessible via a sliding lid, in accordance with another embodiment of the present disclosure;

FIG. 5 is a cross-sectional view of another embodiment of a tissue digester system, illustrating how each of the secondary digestion vessels form agitators and/or baffles within an internal chamber of the rotatable digestion vessel, as well as a different embodiment of a secondary digestion vessel which is freely disposed in the internal chamber of the rotatable digestion vessel;

FIG. 6 is an enlarged cross-sectional view of the tissue digester system of FIG. 5, illustrating the opening of a hinged lid of one of the secondary digestion vessels within the trough, which is independent from the other secondary digestion vessels and independent from the opening of the rotatable digestion chamber;

FIG. 7 is a three-dimensional view of a tissue digester system, in accordance with another embodiment of the present disclosure, wherein the secondary digestion vessels are arranged in the body of the rotatable digestion chamber and the hinged lids open outwards to provide access thereto;

FIG. 8 is a flow diagram of a method of digesting small tissue bodies, which utilizes a tissue digestor system having at least one secondary digestion vessel, in accordance with another embodiment of the present disclosure;

FIG. 9 is a three-dimensional view of a head encasing device which is operable to mechanically facilitate digestion of brain tissue of a deceased body, in accordance with another embodiment of the present disclosure;

FIG. 10 is a cutaway view of the head encasing device of FIG. 9 taken from line A-A, illustrating the head encasing device as fitted to a human head, with the pair of internal projections engaging the craniocervical region anteriorly and posteriorly;

FIG. 11 is a three-dimensional view of the head encasing device of FIGS. 9 and 10 in an open configuration, illustrating the two-part body and relative arrangement of the pair of internal projections; and

FIG. 12 is a flow diagram of a method of facilitating digestion of brain tissue in a head of a deceased body, which utilizes a head encasing device, in accordance with another embodiment of the present disclosure.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this disclosure belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification and claims, the meaning of “a,” “an,” and “the” include plural references unless the context clearly dictates otherwise. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

As indicated above, known devices, systems and methods for the alkaline hydrolysis of biological tissues are limited in several facets. For example, the conventional manner of inserting a caustic substance into a digestion chamber involves inserting fluid, which is typically water, into the digestion chamber and then adding the caustic substance, which is typically in a powdered form, directly to the water. The action of adding the powdered caustic substance directly to the water poses a hazard to a mortician or biocremation operator in that the caustic substance may splash onto the operator, or otherwise react with the water and come into contact with the operator. When the caustic substance is potassium hydroxide (KOH), the reaction with water upon contact is exothermic and the heat released during solvation poses a further risk to the operator. Conventional digestion chamber designs do not allow for the addition of the caustic substance to the digestion chamber when the digestion chamber is closed, or in another manner which protects the operator from the hazard of caustic fluid.

Another limitation of existing devices, systems and methods for the alkaline hydrolysis of biological tissues is that, when it comes to the digestion of smaller tissue bodies (e.g. pets), conventional digestion chambers typically allow for the digestion of several smaller tissue bodies coupled in a single digestion chamber. Such grouped digestion does not allow for the separate retrieval of the undigested remains of the smaller tissue bodies. As such, the sanctity of the remains is not necessarily preserved, particularly where respective families seek the remains of their respective smaller tissue bodies. In an attempt to address this need, efforts have been made at providing physical partitions which fit to the digestion chamber to separate the remains, or otherwise providing reusable permeable bags which are received into the digestion chamber and which can retain therein the smaller remains. However, it has been found that the use of partitions does not effectively prevent comingling of the undigested remains and further, that the removal of the undigested remains is tediously labor intensive.

Existing devices, systems and methods for the alkaline hydrolysis of biological tissues are yet further limited when it comes to the digestion of the heads of bodies. In particular, the cranium encloses the brain in such a manner that it is difficult for digestive fluid to penetrate it. Many conventional digestion chamber designs and methods attempt to address this drawback by increasing the duration of the digestive cycle such that the digestive fluid can eventually enter the cranium through, for example, the eye sockets. Other designs facilitate entry of digestive fluid into the cranium by clamping the head in a fixed position in a stationary digestive chamber and applying a vise-like force to crack it prior to commencing digestion. Others inject digestive fluid directly into the cranium through the eye sockets prior to commencing digestion, whilst yet others aim a recirculation jet at the head to accelerate entry of digestive fluid into the cranium. The former two methods require an additional, time-consuming step, and the latter requires additional equipment, all of which do not fully address the problem and thus leave room for improvement.

The devices, systems and methods described herein provide, in accordance with different embodiments and in general terms only, different examples in which a caustic substance can be safely introduced to a digestion chamber, different examples in which smaller bodies can be retained in separate chambers for digestion and observed prior to easy retrieval of remains, and different examples in which a head encasing device which mechanically facilitates digestion.

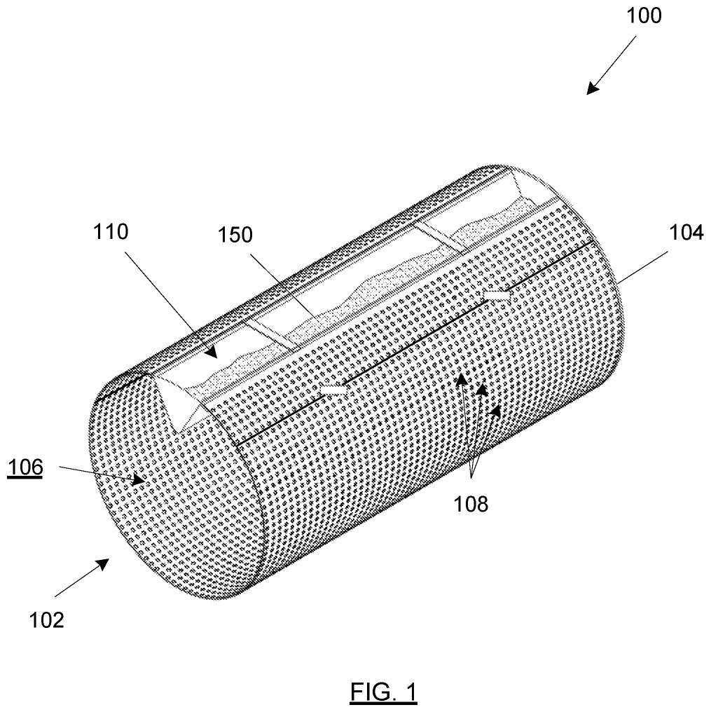

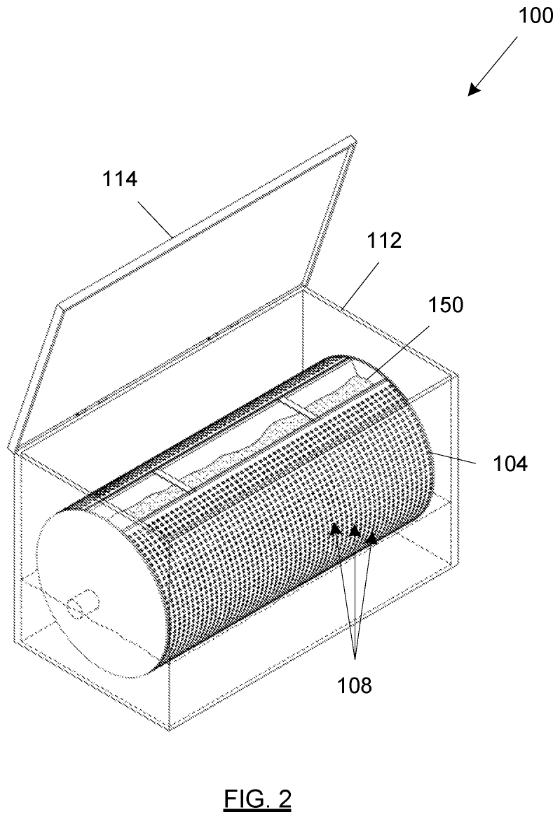

With reference to FIGS. 1 and 2, and in accordance with one aspect of the disclosure, an exemplary embodiment of a tissue digester device, generally referred to using the reference numeral 100, will now be described. The tissue digester device may also be referred to as a “digestion cage”, as similar devices are commonly referred to in the art. The tissue digester device 100 comprises a rotatable digestion vessel 102 having a body 104 which defines an internal chamber 106, a plurality of perforations 108 through the body 104 of the rotatable digestion vessel 102, and a trough 110 formed on the body 104 of the rotatable digestion vessel 102. The trough 110 extends into the internal chamber 106 and is configured to receive a caustic substance 150 when the rotatable digestion vessel 102 is stationary.

In this embodiment, the rotatable digestion vessel 102 is circular in cross-section, as shown, and the internal chamber 106 is also circular in cross-section, matching the shape of the body 104 of the rotatable digestion vessel 102. The body 104 is thus cylindrical in shape and includes two sealed ends (shown in FIG. 2) which provide the rotatable digestion vessel 102. The internal chamber 106 is shaped and dimensioned to receive therein a biological tissue body, such as a cadaver (herein refers to a deceased human body, not restricted to scientific purposes), a carcass (herein refers to a deceased animal, not restricted to one for meat or cooking), or a plurality thereof, for digestion by means of alkaline hydrolysis. The process and parameters of alkaline hydrolysis for the digestion of biological tissue is well-documented and is thus undiscussed for the sake of brevity. The rotatable digestion vessel 102 is capable of withstanding the alkaline hydrolysis process without deterioration, to retain therein any undigested tissue remains after a digestive cycle. In this particular embodiment, the rotatable digestion vessel 102, including the trough 110 which is formed therein, is manufactured of steel. Steel is resistant to corrosion and therefore allows the tissue digester device 100 to be reused in a plurality of digestive cycles, despite the caustic nature of the digestive fluid.

In this embodiment, the plurality of perforations 108 are arranged through the body 104 at spaced apart positions, as shown. The plurality of perforations 108 are sized and/or numerically sufficient to allow digestive fluid to flow into and egress out of the rotatable digestion vessel 102 with ease during a digestive cycle. Such fluid motion through the plurality of perforations 108 is continued even when the digestive fluid becomes saturated with salts and/or amino acids, as a digestive cycle progresses.

In this embodiment, the trough 110 (also referred to as a “chemical trough”) is in the form of a v-shaped notch. As best shown in FIG. 1, the trough 110 is formed in the body 104 of the rotatable digestion vessel 102, and extends or protrudes into the internal chamber 106. More specifically, the trough 110 is formed by two walls which draw together at a point within the internal chamber 106. The internal walls and point of the trough 110 can thus engage a biological tissue body held within the internal chamber 106, as described further below. The trough 110 is impervious to the caustic substance (and specifically, is devoid of perforations or apertures in this embodiment), which enables it to receive the caustic substance 150 when facing upwards, without it immediately egressing out or into the internal chamber 106. In this embodiment, the trough 110 extends along a length of the body 104. Whilst other configurations are contemplated, as discussed below, such elongation of the trough 110 may ensure initial even distribution of the caustic substance 150 in fluid contained in a digestion chamber 112 (see FIG. 2) when a digestion cycle is commenced.

In this embodiment, the tissue digester device 100 further comprises a lid (shown only in FIG. 1) which when opened, provides access to the internal chamber 106. The lid is specifically located in the body 104 around the trough 110, in this embodiment. The lid of the rotatable digestion vessel 102 is a hinged lid which can be securely closed with a fastening mechanism (not shown). As shown, in this embodiment the body 104 further includes strengthening struts which stretch across the trough 110 at spaced apart positions and which can act as handles for opening the lid of the rotatable digestion vessel 102. It is to be appreciated that the lid of the rotatable digestion vessel may be provided at any location on the body, including on the ends of the rotatable digestion vessel, and may be provided with any other suitable opening mechanism (e.g. sliding lid).

As specifically shown in FIG. 2, the tissue digester device 100 in this embodiment is shaped and dimensioned to be receivable into a digestion chamber 112. The tissue digester device 100 can be arranged in the digestion chamber 112 such that the caustic substance 150 is insertable into the trough 110 without directly striking fluid held in the digestion chamber 112. More specifically, the trough 110 can be arranged to face upwards such that the caustic substance 150 can be easily inserted (and without any splashing when, for example, the digestion chamber 112 is already loaded with water or other fluid). In some embodiments, the tissue digester device 100 comprises a marker, guide, spacer, clasp or the like (not shown), which marks the position or otherwise holds the tissue digester device 100 in the digestion chamber 112 such that the trough 110 faces upwards. In this embodiment, the digestion chamber 112 further comprises a lid 114 which provides access to the digestion chamber 112. The lid 114 is a hinged lid which is closed prior to commencing a digestive cycle to create a seal with the digestion chamber 112. The lid 114 of the digestion chamber 112 further prevents the caustic substance or the digestive fluid from coming into contact with a mortician or biocremation operator once rotation of the rotatable digestion vessel 102 commences. Although not shown, the digestion chamber 112 may further comprise a releasable locking mechanism, arranged to releasably lock the digestion chamber 112 during a digestive cycle.

In this embodiment, as best shown in FIG. 1, the trough 110 extends into the internal chamber 106 to form an agitator (or otherwise, projection) within the internal chamber 106. The trough 110, and specifically its internal walls which draw to a point (i.e. a sharp edge), are operable as an agitator for the biological tissue when the tissue digester device 100 is in use. In particular, when biological tissue is loaded into the internal chamber 106 of the rotatable digestion vessel 102 and the rotatable digestion vessel 102 is rotated, the internal walls and/or sharp edge of the trough 110 may come into (continued) contact with a surface of the biological tissue, thereby agitating, palpitating and/or abrading it, to assist in digestion. Initially, this agitation may be of the skin or fur of the biological tissue and later, the agitation may be of the subcutaneous tissue (i.e. fat) and/or muscle fibers of the biological tissue, until the bone of the biological tissue may be reached. Whilst this agitation is generally considered physical agitation, it is to be appreciated that such agitation may expose more biological tissue to the digestive fluid over time, thereby assisting in chemical agitation too. It is to be appreciated, furthermore, that the rotation of the rotatable digestion vessel 102, as described below, in turn rotates the trough 110, thereby continuously contacting and agitating the biological tissue at least at a bottom of the rotatable digestion vessel 102. Furthermore, in embodiments where the tissue digester device 100 is subjected to other movements, such as shaking or rocking, such movements may further increase contact of the biological tissue with the trough 110 and its sharp edge, thereby to increase agitation and/or palpitation, aiding digestion of the biological tissue.

In addition or in alternative to the above, the trough 110 which extends into the internal chamber 106 may also form a baffle within the internal chamber 106. In particular, the internal walls which draw to a point (i.e. sharp edge) may be operable as a baffle for the digestive fluid in the digestion chamber 112 in use. In particular, when the digestion chamber 112 contains digestive fluid, the internal walls of the trough 110 may come into contact with the digestive fluid contained therein, thereby moving it or directing its flow within the internal chamber 106. Otherwise, when the digestion chamber 112 contains water and the caustic substance is to be mixed therewith to create the digestive fluid, the internal walls of the trough 110 may assist in mixing the digestive fluid to solvate the caustic substance. It is to be appreciated that the external walls of the trough 110 may have similar functionality as a baffle, directing fluid flow within the digestion chamber 112.

In this embodiment, although not illustrated to simplify the drawings, the digestion chamber 112 (FIG. 2) comprises a rotation mechanism and the tissue digester device 100 is connectable to the rotation mechanism of the digestion chamber 112 such that it can be rotated during digestive cycles. In some embodiments, the tissue digester device 100 includes central shafts (see FIG. 2) on either end of the body 104, which are aligned and form an axis of rotation through the tissue digester device 100, the central shafts being connectable to the rotation mechanism. In use, rotational movement of the rotatable digestion vessel 102 within the digestion chamber 112 during a digestive cycle mixes the caustic substance 150 with fluid in the digestion chamber 112 to form a digestive fluid (or otherwise supplement it). The trough 110 may also assist in mixing the caustic substance 150 with the fluid, as mentioned. In this embodiment, the caustic substance 150 is KOH and the fluid is water. The mixing thereof by the rotational movement of the rotatable digestion vessel 102 and/or the trough 110 dissolves the KOH 150 in the water to form a solvation which is the digestive fluid. In some embodiments, the rotation mechanism of the digestion chamber 112 is directionally variable such that the direction of rotation of the tissue digester device 100 can be changed either during a digestive cycle or when a digestive cycle is paused, for example from a forward rotation to a reverse rotation, or vice versa. Such variable rotational direction may facilitate agitation (and thus, digestion) of the biological tissue body in use. It is to be appreciated that a speed of rotation of the tissue digester device 100 may also be variable through adjustments to the rotation mechanism, and that variable speeds may also facilitate agitation (and thus, digestion) of the biological tissue body in use. The speed and direction of rotation may be variable, for example, based on a stage in the digestive cycle. In this embodiment, the digestion chamber 112 is specifically a non-pressurized vessel which operates as atmospheric pressure, rendering it simpler to build, maintain and safer to operate.

Therefore, the tissue digester device 100 described with reference to FIGS. 1 and 2 exhibits various improvements over conventional digestion cages. For example, the provision of the trough 110 on the body 104 of the rotatable digestion vessel 102 allows for safer introduction of the caustic substance 150 to the digestion chamber 112 after the fluid (e.g. water) is added. In particular, the caustic substance 150 is held in the upwards facing trough 110 and is only mixed with the fluid once the lid 114 of the digestion chamber 112 is securely closed (sealed) and rotation of the tissue digester device 100 commences (thus, providing indirect introduction thereof). This may, for example, avoid the hazard of the caustic substance 150 and/or the caustic digestive fluid formed splashing upwards towards the mortician or biocremation operator, or otherwise out of the digestion chamber 112. Therefore, in some embodiments, the tissue digester device 100 may provide a safer mechanism of introducing the caustic substance 150 and forming (or supplementing) the digestive fluid in use.

In addition or in alternative to the above, the tissue digester device 100 may improve digestion of any biological tissue contained within the rotatable digestion vessel 102. For example, the trough 110 on the body 104 may provide an internal agitator within the rotatable digestion vessel 102, which increases agitation, palpitation, and/or abrasion of the biological tissue being digested. This may be particularly so where the trough 110 is in the form of a v-shaped notch, having a sharp edge which engages the tissue body as it tumbles and/or rotates within the rotatable digestion vessel 102. Using one embodiment of the tissue digester device 100 with a v-shaped notch as the trough 110, the inventor measured an improved digestive efficacy of 25% (i.e. thus shorter digestive cycle times). More specifically, the average digestive cycle time required to reach sufficiently digested tissue (based on the undigested tissue residue remaining) was reduced from four (4) hours to three (3) hours, at a temperature below boiling (approximately 96° C. or 205° F.) in a non-pressurized system.

Yet further, the tissue digester device 100 may improve digestion of any tissue contained within the rotatable digestion vessel 102 by facilitating fluid flow of the digestive fluid. For example, the trough 110 on the body 104 may provide an internal baffle within the rotatable digestion vessel 102, which increases fluid flow within the digestion chamber 112, largely in a rotational flow (or vortex). The trough 110 may act as a baffle when mixing the digestive fluid too. Increased fluid flow, in turn, may contribute to the digestion of the biological tissue by continuously exposing it to “fresh” digestive fluid.

Various alternative embodiments of the tissue digester device are contemplated to fall within the scope of the present disclosure, some of which are briefly discussed hereunder without limitation.

In other embodiments, the rotatable digestion vessel may be any one of: triangular, circular, square, polygonal or the like in cross-section, with the internal chamber either matching or differing from the rotatable digestion vessel in cross-sectional shape.

In other embodiments, the body of the rotatable digestion vessel may be formed of mesh, the plurality of perforations thereby provided as apertures between the mesh. In other embodiments, the plurality of perforations may be in the form of slots, or a combination of slots and apertures, provided fluid flow of digestive fluid through the body of the rotatable digestion vessel is permitted.

In other embodiments, the trough may comprise only a partially impervious portion, which receives the caustic substance, a remaining portion of the trough including, for example, perforations for fluid flow therethrough. In other embodiments, the trough may extend along only a portion of the body of the rotatable digestion vessel, provided the trough is sized to receive the caustic substance or at least a portion of the caustic substance therein. Furthermore, in other embodiments, the trough may have a cross-section which is circular, square, polygonal, or the like, provided the trough forms a receiving portion in which a caustic substance can be received (typically in the body of the rotatable digestion vessel). In yet other embodiments, the tissue digester device may include a plurality of spaced-apart troughs provided at spaced apart positions and adapted to receive portions of the caustic substance therein.

In other embodiments, the trough may include a projection (or a plurality thereof) which is operable to pierce or penetrate biological tissue contained within the internal chamber of the rotatable digestion vessel. Such piercing or penetration may be beneficial, for example, in facilitating digestion of the biological tissue.

In some embodiments, the rotatable digestion vessel comprises a lid which is adapted to provide access to the internal chamber. In some of these embodiments, the trough may be formed within the lid of the rotatable digestion vessel. In this regard, it is to be appreciated that the caustic substance is typically inserted into the trough after the biological tissue has been loaded into the rotatable digestion vessel. Thus, the lid of the rotatable digestion vessel can be closed once the biological tissue is loaded therein and the caustic substance can be added to the trough before a digestive cycle commences. The lid of the rotatable digestion vessel, in which the trough may be formed, may be removable lid, a hinged lid, a sliding lid or the like. Otherwise, the trough may be formed within the body of the rotatable digestion vessel, adjacent or apart from the lid of the rotatable digestion vessel, such that the lid can be open/closed at any time regardless of whether or not the caustic substance has been inserted in the trough.

In other embodiments, the digestion chamber may be a pressurized vessel.

The digestion chamber may be configured to apply other movements to the rotatable digestion vessel (i.e. in addition to rotation), such as a rocking or see-saw movement of the rotatable digestion vessel, either laterally or longitudinally. In some embodiments, the digestion chamber and/or the rotatable digestion vessel may include a pivot point at which the digestion chamber and/or the rotatable digestion vessel can pivot in two or more directions. In other embodiments, the digestion chamber may be configured to shake or vibrate the rotatable digestion vessel in addition to rotation to increase agitation of the biological tissue, for example. In yet other embodiments, the digestion chamber itself may be configured to rotate, rock or see-saw, shake or vibrate, such that the rotatable digestion vessel contained therein is exposed to the same agitating movement.

In other embodiments, the tissue digester device may be configured to apply vibrational or ultrasonic forces to any one or combination of the device, the biological tissue, or the digestive fluid, to aid in digestion of the tissue body. In one embodiment, the tissue digester device may be configured to apply vibrational or ultrasonic forces to the trough formed in the body of the rotatable digestion vessel. In another embodiment, the tissue digester device may be configured to apply vibrational or ultrasonic forces to one or more baffles or agitators arranged within the rotatable digestion vessel. In a further embodiment, the tissue digestor device may further comprise an ultrasonic probe which can be arranged within or outside of the rotatable digestion chamber to apply ultrasonic waves to any one of the rotatable digestion vessel (or a specific part thereof), the digestion vessel, the tissue body itself, or the digestive fluid.

In other embodiments, the tissue digester device and/or the digestion chamber may further comprise a movement controller which is operable to control movements of the tissue digester device and/or the digestion chamber in accordance with, for example, preset movements of a digestive cycle. To provide a nonlimiting example, preset movements may comprise intermittent forward and reverse rotations, with continuous vibrations and longitudinal pivoting for predetermined periods, where such movement has been determined to facilitate digestion of biological tissue contained in the tissue digester device.

In other embodiments, the tissue digester device may comprise additional projections within the internal chamber of the body of the rotatable digestion vessel. These additional projections may project from an internal wall of the body, or otherwise may be releasably attached to the internal wall of the body. In some embodiments, the additional projections may be bolted to an internal support frame of the rotatable digestion vessel, including axially configured shafts (including a drive shaft, or any in direct or indirect connection with a drive shaft). The additional projections may provide baffles arranged on the internal wall to facilitate fluid flow of the digestive fluid when the device is in use. In addition or in alternative thereto, the additional projections may provide agitators which increase agitation, palpitation and/or abrasion of the tissue contained therein, thereby to reduce the time of the digestive cycle required to sufficiently digest the biological tissue. In some embodiments, the additional projections may be in the form of rod-shaped projections or paddle-shaped projections manufactured of steel.

In other embodiments, the tissue digester device may be manufactured of any one or combination of materials, typically metals, which are resistant to caustic corrosion. In some embodiments, the body of the rotatable digestion vessel may be manufactured of a material distinct from the trough.

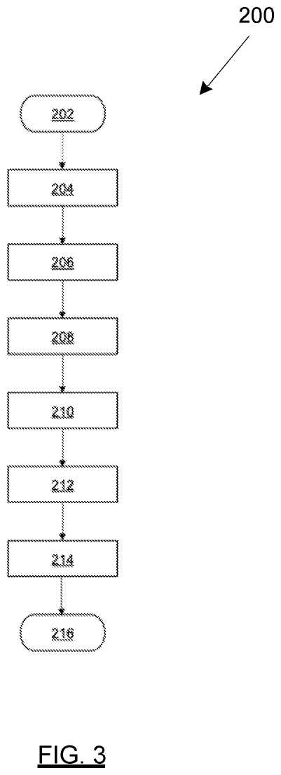

With reference to FIG. 3, and in accordance with another as aspect of the disclosure, an exemplary method of introducing a caustic substance to a digestion chamber, generally referred to using the reference numeral 200, will now be described. The method 200 of introducing the caustic substance to the digestion chamber commences at reference numeral 202.

At 204, the method 200 comprises providing a digestion chamber having a rotatable digestion vessel arranged therein, the rotatable digestion vessel having a plurality of perforations and a trough formed therein. For the purposes of illustration, in this embodiment the rotatable digestion vessel comprises a tissue digester device 100 as described with reference to FIGS. 1 and 2 above. It is to be appreciated, however, that limitation of the present method 200 to such a design of the tissue digester device is not intended by the inventor. At 204, the method 200 may further comprise connecting the digestion chamber to a power supply, such that rotation of the rotatable digestion vessel by the rotation mechanism is electrically powered when a digestive cycle is commenced.

At 206, the method 200 comprises preloading the rotatable digestion vessel with biological tissue which is at least partially digestible. In this embodiment, the biological tissue is any one or more of a cadaver or a carcass, which is to be at least partially digested by means of alkaline hydrolysis. Once the biological tissue is loaded into the rotatable digestion vessel, the lid of the rotatable digestion vessel is closed and secured. It is to be appreciated that the mineral constituents of teeth and bones of vertebrates may not be fully digestible, but that any undigested solid remains or residue which are retained in the closed rotatable digestion vessel may form byproducts with alternative uses. For example, since undigested remains are sterile, it can be crushed into a powder to be utilized as a soil fertilizer or additive, with bone meal providing one particular example.

At 208, the method 200 comprises arranging the rotatable digestion vessel, which in this embodiment is preloaded with the biological tissue, within the digestion chamber such that the trough faces upwards. The rotatable digestion vessel may be rotated manually to a catch-point or marker, or otherwise rotated by automation to a point where the trough faces upwards, dependent on the design of the digestion chamber. In embodiments where the trough is provided in the lid of the rotatable digestion vessel, such arranging of the rotatable digestion vessel may not be necessary since the trough will likely be facing upwards as a result of the preloading of the biological tissue therein. It is to be appreciated that arranging the trough facing upwards prior to inserting any fluid should ensure that the surface area of the trough is at least substantially dry (thus avoiding any potential chemical reaction with the caustic substance once inserted, as described below).

At 210, the method 200 comprises inserting fluid in the digestion chamber. In this embodiment, the fluid comprises water. The water is inserted into the digestion chamber via a fluid inlet arranged on a side of the digestion chamber. The amount of water added may depend on several factors, as recognizable by those skilled in the art, including for example the weight and mass of the biological tissue to be digested, the operating temperature and optionally, if part of the system design, the operating pressure of the digestion chamber during the digestive cycle.

At 212, the method 200 comprises inserting the caustic substance into the upwards-facing trough whilst the rotatable digestion vessel is stationary. By insertion of the caustic substance into the trough (which is at least substantially dry), and not directly into the fluid (i.e. water), method 200 avoids a potentially hazardous scenario wherein the caustic substance may splash onto the mortician or biocremation operator or otherwise exothermically react with the fluid and come into contact with the operator. In this embodiment, the caustic substance comprises potassium hydroxide (KOH), which is in a powdered form. The KOH is inserted into the upwards-facing trough with a suitable dispenser (e.g. steel spoon) in an amount dependent on several factors, as recognizable by those skilled in the art and including, for example, those factors mentioned above. In some embodiments, at 212 the method 200 may comprise measuring an amount of the caustic substance required to achieve a final concentration of digestive fluid required for the digestive cycle. Once the KOH has been inserted, the lid of the digestion chamber (in which the rotatable digestion vessel is arranged) is closed and secured.

At 214, the method 200 comprises rotating the rotatable digestion vessel such that the caustic substance egresses from the trough into the digestion chamber. As noted, the rotatable digestion vessel is only rotated once the digestion chamber is closed (and/or sealed), thus providing a safer mechanism of bringing the caustic substance into contact with the fluid. In this embodiment, the rotating of the rotatable digestion vessel mixes or solvates the caustic substance with the fluid to form a digestive fluid, which in turn is operable to digest the biological tissue in the rotatable digestion vessel (entering the rotatable digestion vessel through the perforations). In this embodiment, the rotating of the rotatable digestion vessel at 214 forms part of a digestive cycle performed by the digestion chamber and rotation is automatically-driven. In addition to rotating, the digestive cycle may include the application of heat by a heater (to heat the digestive fluid) to the digestion chamber which facilitates digestion by means of alkaline hydrolysis, as is known to those skilled in the art. Pressure may also be optionally applied. It is to be appreciated, therefore, that in some embodiments, the method 200 allows some or all of the caustic substance to be mixed with the fluid in a somewhat delayed manner, or released after a given delay from the beginning of the operation of the system, wherein the rotatable digestion vessel is only rotated once the digestion chamber is closed and the caustic substance only then mixes with the fluid (despite being inserted into the trough earlier) to form caustic digestive fluid.

If it becomes necessary to increase or supplement the concentration of the digestive fluid in the digestion chamber after initial operation, the rotating of the rotatable digestion vessel can be paused, the rotatable digestion vessel can be opened, more of the caustic substance can be inserted into the trough (facing upwards, suitable arranging of the rotatable digestion vessel may or may not be necessary), the rotatable digestion vessel can be closed, and rotation of the rotatable digestion vessel can be commenced again to mix the caustic substance with the digestive fluid to further supplement it (or increase concentration). Put differently, any one or combination of steps 208 to 214 can be repeated if the digestive fluid is to be increased or supplemented to increase concentration of the caustic substance in the fluid for the purposes of digestion of the biological tissue.

The method 200 terminates at reference numeral 216 in this embodiment.

Various alternative embodiments of the method of introducing the caustic substance to the digestion chamber are contemplated to fall within the scope of the present disclosure, some of which are briefly discussed hereunder without limitation.

In other embodiments, structural variations of the digestion chamber and the rotatable digestion vessel (including the perforations and the trough) are contemplated, which would be workable in the method. In particular, the trough may take any shape and dimensions, provided retention of the caustic substance is possible whist the rotatable digestion chamber is stationary. Various shapes of rotatable digestion vessel are workable, and the perforations may take the form of slots, apertures, mesh or combinations thereof which permit fluid flow therethrough.

In other embodiments, the fluid may comprise water with additives, or another digestive fluid suited to alkaline hydrolysis. In other embodiments, the fluid may be inserted in the digestion chamber with a hose or via another external fluid supply. In other embodiments, steps 208 and 210 may be interchanged, with the fluid being inserted into the digestion chamber prior to arranging the rotatable digestion vessel such that the trough faces upwards.

In other embodiments, the caustic substance comprises any caustic substance which when mixed with a fluid (i.e. water or otherwise) forms a digestive fluid suited to digestion of at least soft body tissue (e.g. by alkaline hydrolysis). In other embodiments, the caustic substance comprises sodium hydroxide (NaOH). In yet other embodiments, the caustic substance comprises a mixture of KOH and NaOH. The caustic substance may take any form, including powder form, flake form, pellet form, concentrated liquid form, or the like.

In other embodiments, the rotatable digestion vessel may comprise a secondary digestion vessel and the method may comprise preloading the secondary digestion vessel with biological tissue. In such embodiments, the biological tissue in the secondary digestion vessel may be digestible in conjunction with biological tissue in the rotatable digestion vessel. In some embodiments, the biological tissue loaded in the secondary digestion vessel may be kept separate from the biological tissue in the rotatable digestion vessel throughout the digestion cycle for easy removal upon sufficient digestion.

It is to be appreciated that the method may be repeated for the digestion of several biological tissues (or bodies). Accordingly, the method may further comprise stopping a digestive cycle, discharging the digestive fluid from the digestion chamber, removing residual undigested remains (e.g. bone fragments or teeth) from the rotatable digestion vessel (and/or the secondary digestion vessel, if applicable), and repeating the earlier steps of the method for a subsequent biological tissue(s). As mentioned, residual undigested remains are sterile and may have alternative uses, such as in fertilizer applications. Otherwise, where sanctity of remains is less of a priority, the same digestive fluid may be utilized for the digestion of several biological tissues over several digestive cycles.

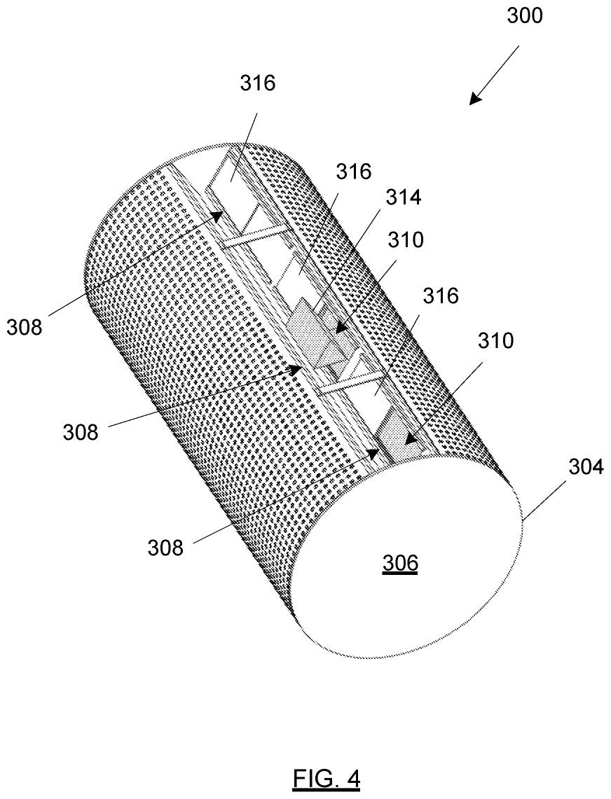

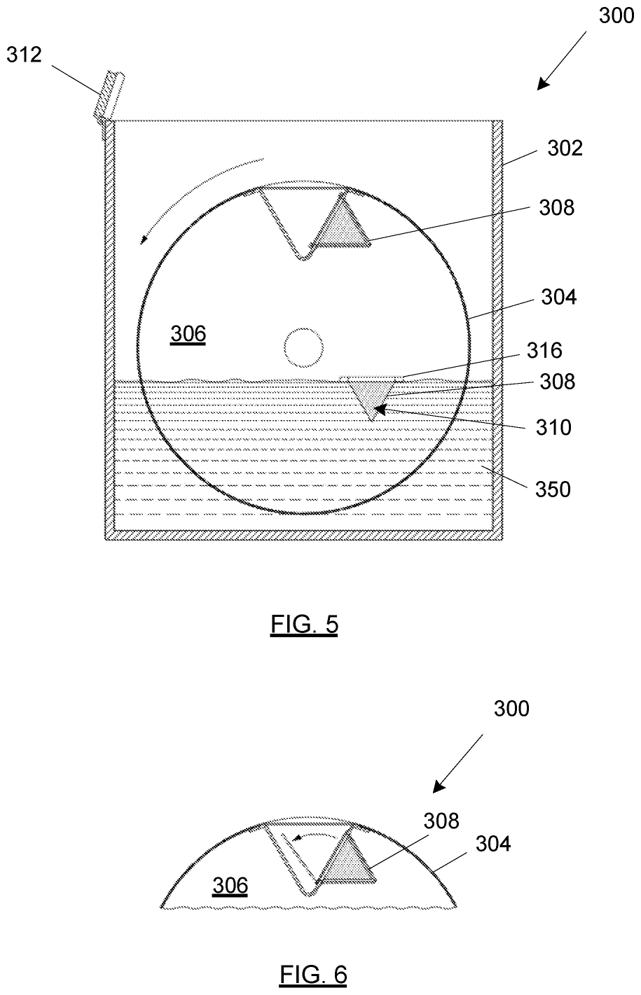

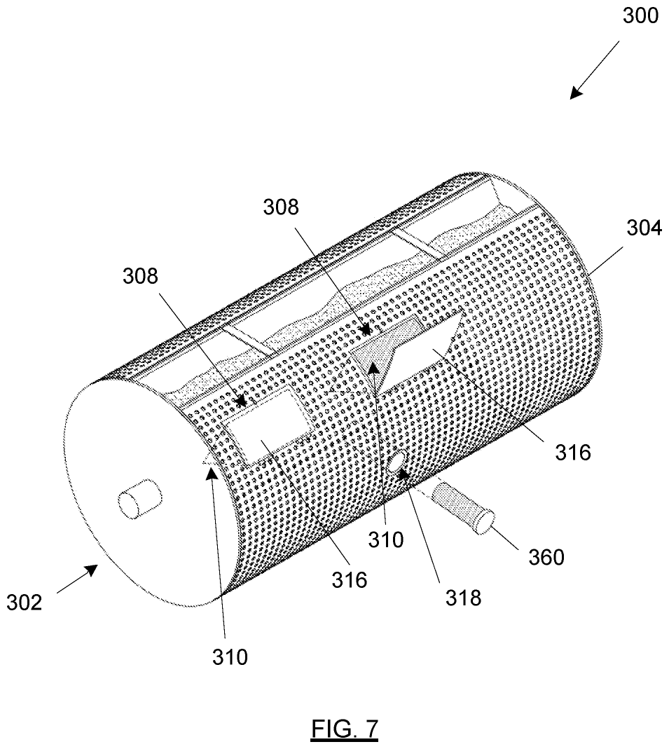

With reference to FIGS. 4 to 7, and in accordance with another aspect of the disclosure, two exemplary embodiments of a tissue digester system, generally referred to using the reference numeral 300, will now be described. The tissue digester system 300 comprises a digestion chamber 302 (shown in FIG. 5) configured to contain a digestive fluid 350, a rotatable digestion vessel 304 (best shown in FIG. 4) arranged within the digestion chamber 302 having a substantially perforated body which defines an internal chamber 306 adapted to receive a large tissue body (not shown) for digestion, and at least one secondary digestion vessel 308 disposable within the internal chamber 306 of the rotatable digestion vessel 304, having a substantially perforated body which defines a secondary internal chamber 310 adapted to receive a smaller tissue body (not shown) for digestion. In use, the at least one secondary digestion vessel 308 moves in conjunction with the rotatable digestion vessel 304 such that the large tissue body and the smaller tissue body are simultaneously at least partially digested through contact with the digestive fluid 350 in the digestion chamber 302. One exemplary embodiment of the tissue digester system 300 is shown in FIG. 4, another in FIGS. 5 and 6, and a third in FIG. 7.

As shown FIG. 5, the digestion chamber 302 is a non-pressurized chamber which is insulated for retaining heat in use. The digestion chamber 302 is largely manufactured of stainless steel and further comprises a lid 312 which provides access to the digestion chamber 302, and seals it when in a closed configuration. It is to be appreciated, however, that alternative shapes and designs of the digestion chamber 302 will be workable in other embodiments. In use, the digestion chamber 302 receives and retains therein a fluid 350, such as water. In FIG. 5, the fluid 350 is shown to only partially fill the digestion chamber 302, however it is to be appreciated that the level or volume of fluid 350 may fill the digestion chamber 302 or otherwise may vary based on, for example, the amount of biological tissue to be digested.

In all three embodiments, the rotatable digestion vessel 304 is a steel drum which is perforated. The internal chamber 306 which is defined by the rotatable digestion vessel 304 has a volume defined by an internal surface area thereof. The internal chamber 306 is shaped and dimensioned to receive the large tissue body which in different use cases, without limitation, can be a cadaver, a carcass or combinations thereof. In all three embodiments, the rotatable digestion vessel 304 is connectable to a rotation mechanism (not shown) which is configured to rotate the rotatable digestion vessel 304 along a central axis of rotation, in both forward and reverse directions, as directed by a movement controller (also not shown).

In these three embodiments, the rotatable digestion vessel 304 includes a trough for inserting a caustic substance (e.g. KOH and/or NaOH, shown in FIG. 7) into the digestion chamber 302 without striking fluid (e.g. water) contained therein. The caustic substance is held in the trough of the rotatable digestion vessel 304 and only mixes with the fluid when the rotatable digestion vessel 304 rotates, thereby forming (or supplementing) the digestive fluid for digestion of the large and smaller tissue bodies. Since the at least one secondary digestion vessel 308 is disposable within the internal chamber 306 of the rotatable digestion vessel 304, these moving together simultaneously in use, only a single rotation mechanism associated with the rotatable digestion vessel 304 is required to move the rotatable digestion vessel 304 and the at least one secondary digestion vessel 308. Furthermore, the at least one secondary digestion vessel 308 does not require a trough of its own, since the perforations in the rotatable digestion vessel 304 and the at least one secondary digestion vessel 308 allow free fluid flow of the digestive fluid into and out of both parts.

In these embodiments, the provision of the rotatable digestion vessel 304 and the at least one secondary digestion vessel 308 to form independent internal chambers in the tissue digester system 300 may facilitate the simultaneous digestion of larger and smaller tissue bodies, whilst retaining the undigested remains of each body separate for independent retrieval post digestion. Put differently, the undigested remains of different bodies can be extracted or retrieved from the tissue digester system 300 separately, preserving perceived sanctity of the individual remains (despite simultaneous digestion). Simultaneous digestion may afford one or more advantages including, for example, increased throughput, reduced turnaround times, decreased cost, reduced digestive fluid wastage or the like, to name a few without limitation.

In the embodiment shown in FIG. 4, the at least one secondary digestion vessel 308 specifically comprises three secondary digestion vessels 308 arranged at different positions within the internal chamber 306 of the rotatable digestion vessel 304. More specifically, the secondary digestion vessels 308 are positioned along a length of an interior wall of the trough of the rotatable digestion vessel 304. The secondary digestion vessels 308 are triangular in cross-section, defining generally triangular prism-shaped secondary internal chambers 310. These respective secondary internal chambers 310 are, in turn, shaped and dimensioned to receive therein respective digestion cannisters 314. The respective digestion cannisters 314 are, in turn, shaped and dimensioned to receive therein smaller tissue bodies for digestion. The secondary digestion vessels 308 are substantially perforated, as shown, allowing fluid to freely flow into and egress out of the secondary internal chambers 310 defined thereby. The secondary internal chambers 310 are accessible via sliding lids 316 defined in the wall of the rotatable digestion vessel 304. The sliding lids 316 are adjustable between an open position (see the lower two secondary digestion vessels 308 in FIG. 4), in which the digestion cannisters 314 can be inserted or removed from the secondary internal chambers 310, and a closed position (see the uppermost secondary digestion vessel 308 in FIG. 4), in which the digestion cannisters 314 are securely retained in the secondary digestion vessels 308 for the duration of the digestive cycle. When the lids 316 are closed, they form an additional barrier which retains the undigested remains in the secondary digestion vessels 308 (i.e. in addition to the container created by the digestion cannisters 314).

In this particular embodiment, the digestion cannisters 314 (or containers) are shaped and dimensioned to receive the smaller tissue body or bodies (the digestion cannisters 314 being receivable into the secondary digestion vessels 308, as shown in FIG. 4). The external shape of the digestion cannisters 314 matches the internal shape of the secondary digestion vessels 308, thus having a generally triangular prism shape. The digestion cannisters 314 also comprise lids (now specifically shown) to insert and remove the smaller tissue body or bodies from the digestion canisters 314. In this embodiment, the digestion cannisters 314 each comprise a frame body surrounded by a fine mesh, which allows the digestive fluid 350 to flow freely into and out of the digestion cannisters 314. Use of a digestion cannister 314 may simplify retrieval of the undigested remains, as the digestion cannister 314 can be quickly removed and emptied. Otherwise, digestion cannisters 314 can be easily relocated from one digestive cycle to another, for example where digestion of a larger tissue body is complete but a smaller tissue body was added later and requires further digestion (and can be digested in another digestion chamber in which the digestive cycle is ongoing).

In the embodiment shown in FIGS. 5 and 6, the secondary digestion vessels 308 include one disposable in the trough of the rotatable digestion vessel 304 (herein referred to as the “detachable secondary digestion vessel”) and one freely disposal inside the internal chamber 306 (herein referred to as the “floating secondary digestion vessel”), each of which will now be described.

In this embodiment, the detachable secondary digestion vessel 308 is removably attachable to an inner surface of the rotatable digestion vessel 304 such that it protrudes into the internal chamber 306 and can rotate in conjunction with the rotatable digestion vessel 304. Although not explicitly shown, the detachable secondary digestion vessel 308 is in the form of a basket that is removably attachable to the inner surface with one or more fasteners, such as releasable clips. The removable attachment or detachability of the detachable secondary digestion vessel 308 as part of the system 300 allows the undigested tissue residue to be easily removed when digestion of the smaller digestive body or bodies contained therein are sufficiently digested (also without requiring access to the internal chamber 306), such that digestion of the larger tissue body in the rotatable digestion vessel 304 can proceed. Furthermore, the detachability of the detachable secondary digestion vessel 308 simplifies the retrieval of the undigested tissue residues, since the detachable secondary digestion vessel 308 can be detached and emptied individually by, for example, inversion. Yet further, the detachability of the detachable secondary digestion vessel 308 allows for customization of the tissue digester system 300 generally and between uses. Different shapes and/or sizes of the detachable secondary digestion vessel 308 can be utilized depending on the tissue bodies to be digested. In some embodiments, a detachable secondary digestion vessel 308 can be utilized to facilitate digestion of the larger tissue body, even where digestion of a smaller tissue body is not required, as will become clear from the below discussion. The detachable secondary digestion vessel 308 in this embodiment is covered by a hinged lid, as shown in FIG. 6, which retains any undigested tissue residues therein and which provides quick access thereto.

Referring back to FIG. 5, the floating secondary digestion vessel 308 in this embodiment is freely disposed within the internal chamber 306 such that in use, the floating secondary digestion vessel 308 freely tumbles upon rotation of the rotatable digestion vessel 304. In this embodiment, the floating secondary digestion vessel 308, which is also shaped and dimensioned to receive a smaller tissue body, is formed by a cone-shaped frame body surrounded in a fine mesh. The floating secondary digestion vessel 308 further comprises a floatation pocket (not specifically shown) which provides floatation to the floating secondary digestion vessel 308 when in the digestive fluid 350. The floatation pocket may include a foam pocket, an air pocket or the like, which renders the floating secondary digestion vessel 308 buoyant in the digestive fluid 350. It is to be appreciated that, in use, rotation of the rotatable digestion vessel 304 will cause the floating secondary digestion vessel 308 to tumble, the rotational force thereof typically being greater than the floatation afforded by the floatation pocket. When the rotatable digestion vessel 304 is stationary, however, the floatation provided by the floatation pocket will cause the floating secondary digestion vessel 308 to float to the surface of the digestive fluid 350. In use, floatation of the freely disposed floating secondary digestion vessel 308 may assist in the retrieval thereof from the tissue digester system 300 when the smaller tissue body is sufficiently digested. The floating secondary digestion vessel 308 in this embodiment comprises a lid 316 through which access to the secondary internal chamber 310 is provided, the lid 316 being a hinged lid provided at one end of the floating secondary digestion vessel 308.3.

With reference to FIG. 7, another embodiment of the tissue digester system 300 is illustrated, wherein the secondary digestion vessels 308 are arranged in the body 104 of the rotatable digestion chamber 302 at spaced apart positions (and not in the trough) and the hinged lids 316 open outwards to provide access thereto. In this embodiment, the secondary digestion vessels 308 receive smaller biological tissues directly therein, without the use of digestion cannisters or the like. The undigested remains which are left after digestion can be scraped out or otherwise removed from the secondary digestion vessels 308. This embodiment of the tissue digester system 300 further illustrates an additional agitator projection 360 which can be removably coupled to the body 304 of the rotatable digestion chamber 302. The additional agitator projection 360 is a rod-shaped projection which is received into an aperture 318 formed in the body 304, such that it protrudes into the internal chamber 306. The additional agitator projection 360 may act as an agitator to facilitate digestion of the large tissue body (i.e. by agitation, palpitation, abrasion or penetration) or otherwise may be detached when such agitation is not required, for example, or where more space is required in the internal chamber 306 to accommodate the large tissue body.

In all embodiments described herein, the tissue digester system 300 further comprises a heater (not shown) which is configured to heat the fluid contained in the digestion chamber 302. In one embodiment, the heater is only activated upon commencement of a digestive cycle (with the caustic substance and the fluid already inserted), such that the heat released from the exothermic reaction of the solvating of the caustic substance with the fluid can be accommodated without exceeding the tolerances of the digestion chamber 302 (particularly the seal of its lid 312) and creating a potentially hazardous scenario for mortician or biocremation operators.

In use of these embodiments, the rotatable digestion vessel 304 is loaded with a larger tissue body and the secondary digestion vessels 308 are loaded with smaller tissue bodies (e.g. pets). The digestion chamber 302 is filled with digestive fluid 350 (or it is otherwise formed therein by the addition of components separately which later mix), the lid 312 of the digestion chamber 302 is closed, and a digestive cycle is commenced, applying heat to the digestion chamber 302 whilst the rotatable digestion vessel 304 is rotated, in turn simultaneously moving (i.e. rotating or tumbling) the secondary digestion vessels 308. In some embodiments, where a caustic substance is inserted into the trough, the tissue digester system 300 may be further operable to pause a digestion cycle with the trough facing upwards (using a spacer, guide or marker, for example), or otherwise the rotatable digestion vessel 304 may be manually rotatable until the trough faces upwards, which thus provides easy access to the secondary digestion vessels 308 via the lids 316 formed in the trough. In the use of such embodiments, the smaller tissue body or bodies would typically be inserted in the digestion vessels 308 (or cannisters 314) prior to the insertion of the caustic substance into the trough. Once a digestive cycle has commenced, the caustic substance solvates with the fluid 350 to commence digestion, and when the digestive cycle is paused, the caustic substance is typically fully solvated and does not block access to the secondary digestion vessels 308.

In all embodiments of the tissue digester system 300 described herein, the secondary digestion vessels 308 are all operable in use as baffles and/or agitators within the internal chamber 306 of the rotatable digestion vessel 304. As baffles, the secondary digestion vessels 308 assist in directional flow of the digestive fluid 350 within the rotatable digestion vessel 304 in the digestive chamber 302. In particular, the secondary digestion vessels 308 arranged on the wall of the rotatable digestion chamber 304 protrude into the internal chamber 306 and as the rotatable digestion vessel 304 rotates, those secondary digestion vessels 308 may form blades on the internal surface of the rotatable digestion vessel 304, which move through the digestive fluid. Accordingly, these secondary digestion vessels 308 may contribute to the rotational or vortex flow of the digestive fluid within the digestive chamber 302 (mixing the digestive fluid initially and continuously contributing to fluid flow). As agitators, the secondary digestion vessels 308 assist in agitating the large tissue body contained within the internal chamber 306 of the rotatable digestion vessel 304 by agitating, palpitating and/or abrading a surface area of the large tissue body. In particular, some of the secondary digestion vessels 308 protrude into the internal chamber 306 such that as the rotatable digestion vessel 304 rotates, the secondary digestion vessels 308 make contact with the large tissue body (which tends downwards under the force of gravity) and continuously agitate, palpitate and/or abrade the surface area of the large tissue body, such that digestive fluid 350 can enter or penetrate increased surface area to aid digestion. Furthermore, as the large tissue body tumbles during rotation of the rotatable digestion vessel 304, different parts of the large tissue body will be impacted and agitated, palpitated and/or abraded by the secondary digestion vessels 308. The floating secondary digestion vessel 308, on the other hand, tumbles with rotation of the rotatable digestion vessel 304, thereby continuously colliding with the large tissue body and thus continuously agitating, palpitating and/or abrading the surface area of the large tissue body, such that digestive fluid 350 can enter or penetrate and increased surface area to aid digestion. In embodiments where the secondary digestion vessels 308 include sharp edges, the agitation may be even more pronounced.

Appreciating that the smaller tissue bodies may be sufficiently digested prior to the larger tissue body (i.e. due to less digestible tissue), the digestive cycle may be paused once the smaller tissue bodies are sufficiently digested or otherwise suspected to be so. In such an instance, rotation may be paused, the lid 312 of the digestion chamber 302 may be opened, the lid 316 of the secondary digestion vessel 308 may be opened, the undigested remains (e.g. teeth and bones) removed from the secondary internal chamber 310 of the secondary digestion vessel 308, the lid 316 of the secondary digestion vessel 308 closed, the lid 312 of the digestion chamber 302 may be closed, and the digestive cycle may be restarted, including rotation of the rotatable digestion vessel 304 to further digest the larger tissue body (and any other smaller tissue bodies not yet sufficiently digested). It is to be appreciated that, over-digestion of smaller tissue bodies when combined with a larger tissue body or bodies in a typical three (3) hour digestive cycle results in very little (and sometimes, no) retrievable undigested remains of the smaller tissue body. This can be problematic, for example, where families desire the undigested remains to be returned for ceremonious reasons. Therefore, in some embodiments, the tissue digester system 300 allows for retrieval of the undigested remains of smaller tissue remains from secondary digestion vessels 308 (and/or digestion cannisters 314, as the case may be) individually, preserving sanctity, and/or prior to over-digestion when digested simultaneously with a larger tissue body (i.e. before all smaller tissue body remains are digested).

In addition or in alternative to the above, the provision of the secondary digestion vessels 308 in the form of mesh-like structures in some embodiments, or at least with perforations, allows for the visual inspection of the undigested remains contained therein during the digestive cycle, without necessarily opening the secondary digestion vessels 308 (or the internal chamber 306, for that matter). This may reduce the time required to inspect the digestion process of smaller tissue bodies and may further reduce the risk of over-digestion when simultaneously digested with a larger tissue body. Furthermore, the mesh-like structures of the secondary digestion vessels 308 may retain smaller bone and teeth fragments therein, particularly as opposed to the size of fragments retainable by the rotatable digestion vessel 304. The same advantages may be applicable, and even amplified in some embodiments, where digestion cannisters 314 are used within the secondary digestion vessels 308.

In all embodiments described herein, the secondary digestion vessels 308 are all reusable and are dishwasher safe, and can therefore can be easily cleaned after use in a digestive cycle.

Various alternative embodiments of the tissue digester system are contemplated to fall within the scope of the present disclosure, some of which are briefly discussed hereunder without limitation.

In other embodiments, alternative designs of the rotatable digestion vessel are contemplated which include, for example, different shapes, structures, internal projections, and digestion-aiding movement, without limitation.

In some embodiments, the rotatable digestion vessel comprises at least one wall and the at least one secondary digestion vessel is formed by the at least one wall protruding into the internal chamber. Therefore, in such embodiments, the at least one secondary digestion vessel forms part of or is integral to the rotatable digestion vessel. The at least one secondary digestion vessel in such embodiments may include a hinged or sliding lid, arranged to be flush with an external surface of the at least one wall of the rotatable digestion vessel.

In other embodiments, the at least one secondary digestion vessel may be any one of: triangular, circular, square or polygonal, in cross-section, provided the at least one secondary digestion vessel can receive therein a smaller tissue body or a digestion cannister, as the case may be. In some embodiments, the at least one secondary digestion vessel may be formed by a framed body surrounded by a fine mesh. In some embodiments, the lid of the secondary digestion vessels may be sliding lids, hinged lids, or other known lid designs which provide access to the secondary internal chamber. In other embodiments, the at least one secondary digestion vessel may be in form of a reusable digestive bag which is permeable to digestive fluid. It is to be appreciated that, in different embodiments, the number and arrangement of secondary digestion vessels can vary based on the load to be digested. In a similar vein, different shaped and/or sized secondary digestion vessels can be interchangeably employed depending on the shape and/or size of the biological tissue body to be digested.

In some embodiments, the at least one secondary digestion vessel may include a plurality of agitating projections which extend outwards from an external surface of the secondary digestion vessel into the internal chamber of the rotatable digestion chamber. In other embodiments, the body of the rotatable digestion chamber may include a plurality of additional agitator projections which protrude into the internal chamber at spaced apart positions.

In other embodiments, the tissue digester system may further comprise more than one digestion cannister shaped and dimensioned to receive smaller tissue bodies, each digestion cannister being receivable into the at least one secondary digestion vessel. In some embodiments, two or more digestion cannisters may be receivable into a single secondary digestion vessel. The at least one digestion cannister may be manufactured of or contain any perforated material, such that digestive fluid can enter the cannister to digest the smaller tissue body in use.

In other embodiments, the rotatable digestion vessel and/or the digestion chamber may further comprise movement mechanisms in alternative or addition to the rotation mechanism, to increase agitation of the biological tissue (and thus, accelerate digestion). For example, movement mechanisms may allow for the rocking or see-saw movement of the rotatable digestion vessel and/or the digestion chamber, laterally and/or longitudinally, about a pivot point. In another example, movement mechanisms may allow for the shaking, vibration or application of ultrasonic energy to the rotatable digestion vessel and/or the digestion chamber.

In other embodiments, the digestion chamber may comprise a pressurized digestion chamber which operates at pressures above atmospheric pressure.

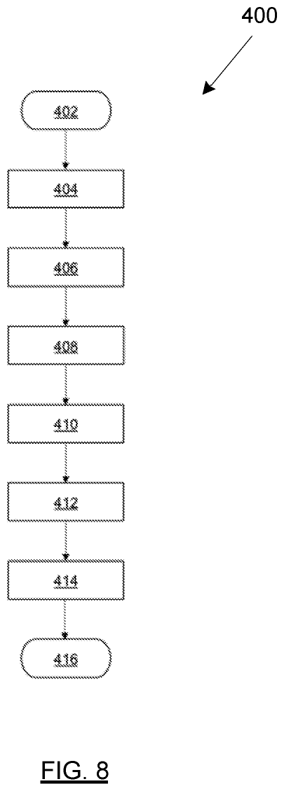

With reference to FIG. 8, and in accordance with another aspect of the disclosure, an exemplary method of digesting small tissue bodies, generally referred to using the reference numeral 400, will now be described. The method of digesting small tissue bodies 400 commences at reference numeral 402.

At 404, the method 400 comprises providing a tissue digester system. The tissue digester system comprises at least a digestion chamber configured to contain a digestive fluid, a rotatable digestion vessel arranged within the digestion chamber having a substantially perforated body which defines an internal chamber, and at least one secondary digestion vessel disposed within the internal chamber of the rotatable digestion vessel having a substantially perforated body which defines a secondary internal chamber. In this embodiment, the tissue digester system specifically comprises a tissue digester system as described with reference to FIGS. 4, 5 and 6, or 7, above. It is to be appreciated, however, that limitation of the present method 400 to such designs of the tissue digester system is not intended by the inventor.

At 406, the method 400 comprises loading a large tissue body into the internal chamber of the rotatable digestion vessel. The large tissue body is to be simultaneously digested with the small tissue body, and typically comprises a cadaver or a large animal body. The large tissue body is typically loaded manually by a mortician or biocremation operator, but may also be loaded with the aid of hoist or other mechanical lift system.

At 408, the method 400 comprises loading a small tissue body to be digested into the secondary internal chamber of the at least one secondary digestion vessel. In this embodiment, the small tissue body comprises a carcass under one kilogram in body weight.

Such carcasses may include, for example and without limitation, deceased pocket-pets such as guinea pigs, hamsters, hedgehogs, mice, rats, gerbils, chinchillas, reptiles, birds, or the like, or otherwise smaller deceased pet animals such as small dogs, juvenile dogs, cats, juvenile cats, rabbits, or the like. In some embodiments, a plurality of small tissue bodies can be loaded into different secondary digestion vessels.

At 410, the method 400 comprises adding digestive fluid to the digestion chamber. In this embodiment, the adding of the digestive fluid comprises separately adding to the digestion chamber water and a caustic substance. The caustic substance may be KOH and/or NaOH, which may be added separately to avoid any potential hazards associated with adding powdered or pelleted caustic substances to water.

At 412, the method 400 comprises rotating the rotatable digestion vessel for a predetermined period to digest the small tissue body through exposure to the digestive fluid in a digestion cycle. In this embodiment, rotation of the rotatable digestion vessel mixes the caustic substance with the fluid to form the digestive fluid. The digestion cycle, in this embodiment, further comprises applying heat which is a known component of the alkaline hydrolysis of biological tissues and which may be adjustable based on the amount of tissue to be digested in the digestive cycle or other relevant parameters. Since this embodiment of the method 400 includes loading a large tissue body into the internal chamber of the rotatable digestion vessel at 406, it is to be appreciated that at 412, the rotation of the rotatable digestion vessel also exposes the large tissue body to the digestive fluid in the digestion cycle, thus digesting it simultaneously with the small tissue body.

At 414, the method 400 comprises pausing the digestion cycle of the tissue digester system to remove undigested remains of the small tissue body in the at least one secondary digestion vessel upon sufficient digestion. The at least one secondary digestion vessel retains the undigested remains therein, separate from the large tissue body, for easy inspection and removal (for further processing whilst the large tissue body digests further). The digestion cycle may be paused at any time to determine whether the small tissue body is sufficiently digested by means of inspection of the undigested tissue remaining in the secondary internal chamber of the secondary digestion vessel. Otherwise, the digestion cycle may be paused at set times, or predetermined times, typically based on known weight of the small body and other digestion cycle parameters (e.g. heat, pressure, concentration digestive fluid or the like) or related calculations.

In this embodiment, the method 400 terminates at reference numeral 416.

Various alternative embodiments of the method of digesting small tissue bodies are contemplated to fall within the scope of the present disclosure, some of which are briefly discussed hereunder without limitation.

In other embodiments, the step of loading a small tissue body to be digested into the secondary internal chamber of the at least one secondary digestion vessel may comprise loading a plurality of small tissue bodies to a plurality of secondary digestion vessels disposable within the rotatable digestion vessel. In such embodiments, the method may enable simultaneous digestion of the plurality of small tissue bodies in the tissue digester system, whilst keeping the undigested remains separate.

In other embodiments, the tissue digester system utilized in the method may comprise at least one digestion cannister receivable into the secondary internal chamber of the at least one secondary digestion vessel. In such embodiments, the step of loading of the small tissue body comprises loading the small tissue body into the at least one digestion cannister, and inserting the at least one digestion cannister into the secondary internal chamber of the at least one secondary digestion vessel. Further in such embodiments, the step of pausing the digestion cycle of the tissue digester system further comprises removing the at least one digestion cannister from the at least one secondary digestion vessel upon sufficient digestion of the small tissue body. Having a separate digestion cannister(s) receivable into the at least one secondary digestion vessel further facilitates rapid removal of undigested remains from the tissue digester system.

In other embodiments, the step of adding the digestive fluid to the digestion chamber may be automated, for example, such that the fluid and the caustic substance are mixed as introduced to the digestion chamber by means of automated machinery.

In other embodiments, the method may comprise the further step of restarting the digestion cycle of the tissue digester system, particularly to continue digestion of small tissue bodies not yet sufficiently digested and/or the large tissue body.

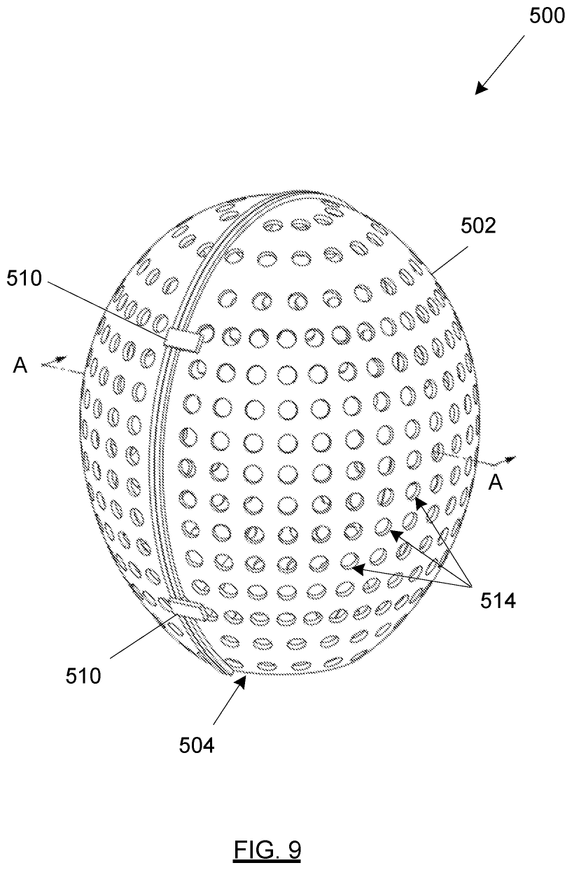

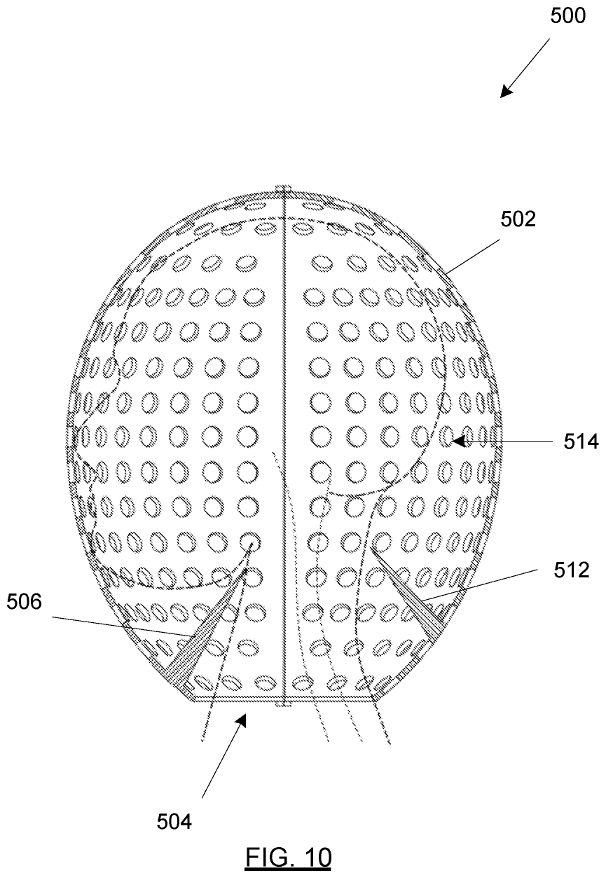

With reference to FIGS. 9 to 11, and in accordance with another aspect of the disclosure, an exemplary head encasing device, generally referred to using the reference numeral 500, will now be described. The head encasing device 500 is operable to mechanically facilitate digestion of brain tissue of a deceased body. The head encasing device 500 comprises a body 502 which is shaped and dimensioned to receive and secure therein a head of the deceased body, an aperture 504 formed in the body 502 which is shaped and dimensioned to surround a neck of the deceased body, and an internal projection 506 extending from an internal surface of the body 502 proximate the aperture 504 to engage a craniocervical region of the head, in use to pierce the craniocervical region such that digestive fluid can enter a cranium of the head to digest the brain tissue.

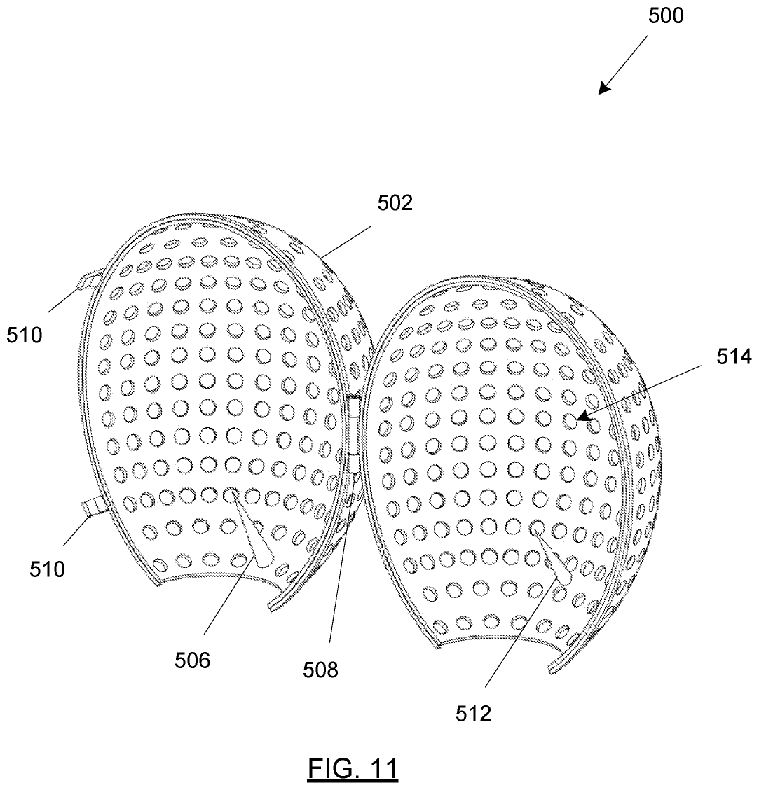

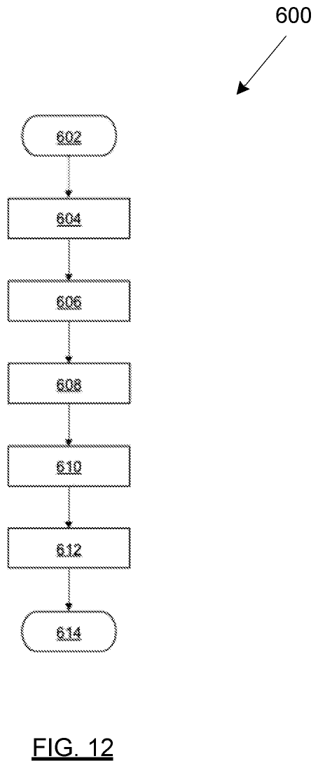

In this embodiment, as best shown in FIG. 10, the head is a human head and the body 502 is substantially ovoid-shaped to fit around the human head. The body 502 specifically comprises a two-part body, as best shown in FIG. 11, and is substantially rigid. The two-part body 502 comprises a hinged connection 508 which allows the two-part body 502 to be opened to receive the human head and closed to surround the human head. The body 502 further comprises releasable fasteners 510 for securing the body 502 in a closed configuration around the head and the neck. In this embodiment, the releasable fasteners 510 are in the form of releasable friction-fit clips.

In this embodiment, the internal projection 506 is arranged to pierce or penetrate the craniocervical region of the body in use, such that the digestive fluid can enter a foramen magnum of the head. Typically, the head encasing device 500 is affixed to the head of the human body when it is loaded within (or otherwise prior to loading within) a rotatable digestion vessel within a digestion chamber (not shown). When a digestive cycle commences, the digestion chamber is filled with digestive fluid and the rotatable digestion vessel rotates, causing the human body to tumble within the rotatable digestion vessel. Such tumbling of the human body causes the head encasing device 500 to move in various directions, often colliding with internal walls of the rotatable digestion vessel. This tumbling and ricochet action may urge the internal projection 506 to pierce the skin proximate the craniocervical region, allowing digestive fluid to penetrate the skin and commence digestion of internal tissue. With further tumbling and ricochet action, the internal projection 506 reaches the foramen magnum, thereby allowing the digestive fluid to enter the cranium for the digestion of brain tissue.

In this embodiment, as shown in both FIGS. 10 and 11, the head encasing device 500 further comprises an opposing internal projection 512 which is arranged substantially opposite the internal projection 506. The internal projection 506 and the opposing internal projection 512 form a pair of internal projections which engage the craniocervical region at an anterior position and a posterior position, respectively. More specifically, the pair of internal projections 506 and 512 comprise a pair of spikes and they are arranged to pierce the craniocervical region anteriorly and posteriorly to reach the foramen magnum in due course, as described. In use, once the pair of spikes 506 and 512 reach the foramen magnum, the puncture formed allows digestive fluid to penetrate the cranium of the human head, to facilitate digestion of the brain tissue. It is to be appreciated that, depending on the body and the digestive cycle, only one of the spikes 506 and 512, or one followed later by the other, may reach the foramen magnum, but that any piercing or penetrating action may facilitate exposure to the digestive fluid.

In this embodiment, the body 502 is at least partially perforated such that it is permeable to the digestive fluid. More specifically, the body 502 includes a plurality of apertures 514 which allow digestive fluid to enter into and egress out of the head encasing device 500. The plurality of apertures 514 are sized and of a sufficient number to not hinder flow of the digestive fluid in the digestion chamber and/or into and out of the head encasing device 500, even when the digestive fluid becomes saturated with salts and/or amino acids as digestion progresses.

In this embodiment, the head encasing device 500 is manufactured of a non-digestible material such that the head encasing device 500 is reusable for a plurality of digestive cycles. More specifically, the body 502 and the pair of spikes 506 and 508 are manufactured of steel.

In use, prior to commencing a digestive cycle, the head encasing device 500 is fitted to the head of a cadaver such that the aperture 504 surrounds the neck of the cadaver and the pair of spikes 506 and 512 are arranged to engage (or almost engage) the craniocervical region of the cadaver anteriorly and posteriorly (see FIG. 10). Once the digestive cycle is commenced, rotation of the rotatable digestion vessel within the digestion chamber (which is not pressurized) urges the pair of spikes 506 and 512 to pierce the skin and in due course, to reach the foramen magnum to create a puncture reaching the brain tissue. The puncture facilitates access of the digestive fluid to the brain tissue in the cranium, thus facilitating digestion thereof. It is to be appreciated that if the head encasing device 500 is dislocated or displaced during the rotation and ricochet motion such that the pair of spikes 506 and 512 are no longer anteriorly and posteriorly positioned, the design of the head encasing device 500 will continue to urge the pair of spikes 506 and 512 upwards to puncture the craniocervical region to facilitate entry of the digestive fluid into the cranium or otherwise facilitate exposure to the digestive fluid.

In use, the head encasing device 500 may further encourage the detachment of the head from the body of the cadaver, specifically due to the deterioration of soft tissue connecting the spinal column to the cranium (i.e. atlas and axis vertebrae), further facilitating digestion of both the head and the neck, respectively. Once the digestive cycle is complete, the head encasing device 500 can be removed from the digestive chamber, opened to remove any undigested residue, washed and reused.

Various alternative embodiments of the head encasing device are contemplated to fall within the scope of the present disclosure, some of which are briefly discussed hereunder without limitation.

In other embodiments, the deceased body may be an animal body, the head being an animal head and the body of the head encasing device being shaped and dimensioned to fit around the animal head.

In other embodiments, the body of the head encasing device may be formed of more than two parts which cooperate to surround the head of the deceased body and hold the internal projection(s) in near engagement with the craniocervical region of the head.

In other embodiments, the releasable fastener may be any one or more of: a clasp, a snap hook fastener, a nut and bolt fastener, a friction fit fastener, or the like, without limitation.

In other embodiments, the head encasing device may further comprise one or more agitator projections which extend inwards from the internal surface of the body. In some embodiments, the one or more agitator projections may be arranged on the internal surface to engage temple regions of the head. More specifically, the one or more agitator projections may be configured to fracture one or both pterions of the cranium on lateral sides of the head. It is to be appreciated that the pterions are the weakest point of the cranium, thus presenting a good candidate for entry to the cranium. The one or more agitator projections may comprise rod-shaped projections, spike projections, paddle-shaped projections, or the like. Typically, the one or more agitator projections may comprise two agitator projections arranged to engage the temple regions of a human head secured inside the head encasing device. In other embodiments, the head encasing device may comprise a plurality of agitator projections arranged around the internal surface of the body at spaced apart regions to engage different regions of the head to agitate the skin and/or fracture the cranium at different regions for digestive fluid entry.

In other embodiments, the head encasing device may include a rough internal surface to cause further agitation and/or abrasion when in contact with the head. For example, the plurality of apertures may be punctured in the body inwards such that the rim of each aperture forms a raised rim against which the head will rub or scrape when in use.

With reference to FIG. 12, and in accordance with another aspect of the disclosure, an exemplary method of facilitating digestion of brain tissue in a head of a deceased body, generally referred to using the reference numeral 600, will now be described. The method 600 commences at reference numeral 602.

At 604, the method 600 comprises loading the deceased body into a rotatable digestion vessel of a tissue digester system. In this embodiment, the deceased body is a cadaver (i.e. deceased human body). Whilst any tissue digester system and any rotatable digestion vessel suitable for alkaline hydrolysis may be utilized, in this embodiment a tissue digester system such as described with reference to FIGS. 4, 5 and 6, or 7 above, is contemplated for illustrative purposes. It is to be appreciated, however, that limitation of the present method 600 to such designs of the tissue digester system is not intended by the inventor. The cadaver is loaded into the stationary and open rotatable digestion vessel (which is perforated), the rotatable digestion vessel being disposed within a digestion chamber which retains digestive fluid in use. The digestion chamber includes a rotation mechanism for rotation of the rotatable digestion vessel. Whilst loading of the deceased body is typically performed by a mortician or biocremation operator, loading with a hoist, a mechanical lift, or other automated loading may be workable in other embodiments.

At 606, the method 600 comprises fitting a head encasing device to the head of the cadaver (in this embodiment, within the rotatable digestion vessel). The head encasing device is fitted to the head such that a pair of internal projections disposed within the head encasing device engage or are in near engagement with a craniocervical region of the cadaver. In this particular embodiment, the head encasing device is a head encasing device as described with reference to FIGS. 9 to 11 above. It is to be appreciated that limitation of the present method 600 to such designs of the head encasing device is not intended by the inventor. However, in this embodiment, the head encasing device comprises a hinged two-part body and the fitting of the head encasing device to the head comprises the mortician or biocremation operator lifting the head of the cadaver, opening the hinged two-part body and inserting the head in one part of the hinged two-part body, and closing another part of the hinged two-part body such that the hinged two-part body is secured to the head. The two parts of the body of the head encasing device are secured together with a releasable fastening means. It is to be appreciated, of course, that other designs of the head encasing device may require different fitment procedures and that fitment to the head prior to insertion into the rotatable digestion vessel may be workable.

At 608, the method 600 comprises inserting digestive fluid into the tissue digester system. In this embodiment, the inserting of the digestive fluid comprises introducing water into the digestion chamber and inserting a caustic substance (KOH and/or NaOH) into a trough of the rotatable digestion chamber. The digestive fluid is only formed once the rotatable digestion vessel rotates, mixing the water and the caustic substance. It is to be appreciated, however, that alternative means of inserting digestive fluid may be workable in different embodiments. In this embodiment, once the water and caustic substance are inserted, the digestion chamber is closed for a digestion cycle to commence (which solvates the caustic substance in the water to form digestive fluid).

At 610, the method 600 comprises rotating the rotatable digestion vessel (in this embodiment, within the digestion chamber) such that the rotation thereof causes the head encasing device to continuously impact or collide with an internal surface of the rotatable digestion vessel, urging the internal projections to pierce or penetrate the craniocervical region to facilitate entry of the digestive fluid into a cranium of the head for digestion of the brain tissue. In particular, as rotation of the rotatable digestion vessel continues, the head encasing device typically ricochets off the internal surface of the rotatable digestion vessel to “bounce” up and down. The rotating of the rotatable digestion vessel and ricocheting of the head encasing device urges the pair of internal projections to pierce or penetrate the craniocervical region to reach a foremen magnum of the cranium. The punctures formed by the pair of internal projections assist in entry of the digestive fluid into the cranium for digestion of brain tissue. In this embodiment, the rotating of the rotatable digestion vessel and ricocheting of the head encasing device further assists in detachment of the head from the deceased body. Detachment may be useful, for example, to stimulate or accelerate digestion of the head and neck regions further, by exposing tissue to the digestive fluid.

It is to be appreciated that at 610, the method may further comprise applying heat and/or pressure to the tissue digester system as part of conventional alkaline hydrolysis digestion process. As is known in the art, heat may be applied until the digestive fluid reaches and is maintained at a temperature of 150° C., and optionally, a pressure of up to approximately 70 psig may be maintained in certain system designs. Variances of operational parameters are expected in different system designs.

At 612, the method 600 comprises pausing rotation of the rotatable digestion vessel when the cadaver is sufficiently digested. In particular, the digestive cycle may be ceased when the deceased body is digested to the point that only indigestible tissue residue (e.g. teeth and bones) remain. The digestive cycle may be paused at set or predetermined times based on expected digestion progress, or otherwise may be paused periodically to visually inspect the digestion progress. If digestion of the cadaver is not yet sufficient at the point of pausing rotation, rotation may be recommenced until the cadaver is sufficiently digested.

In this embodiment, the method 600 terminates at reference numeral 614.

Various alternative embodiments of the method of facilitating digestion of brain tissue in a head of a deceased body are contemplated to fall within the scope of the present disclosure, some of which are briefly discussed hereunder without limitation.

In other embodiments, steps 604 and 606 may be interchanged, such that the head encasing device is fitted to the head and neck of the cadaver (i.e. 606) prior to the cadaver with the fitted head encasing device being loaded into the rotatable digestion vessel of the tissue digester system (i.e. 604). Such embodiments are envisaged where, for example, the space within the rotatable digestion vessel is limited and pre-fitting of the head enclosing device to the cadaver on a washing table is easier for a mortician or biocremation operator.

In some embodiments, the head encasing device may comprise one or more agitator projections arranged on an internal surface thereof, which in use engage temple regions of the head. In such embodiments, the rotating of the rotatable digestion vessel and ricocheting of the head encasing device urges the one or more agitator projections to fracture one or both pterions of the cranium. Otherwise, the one or more agitator projections may serve to agitate, palpitate and/or abrade the head of the cadaver to expose more tissue to the digestive fluid to aid digestion.

In other embodiments, the step of inserting digestive fluid into the tissue digester system may comprise automated insertion of the digestive fluid, particularly where the digestion chamber and/or system provides such functionality.

In other embodiments, the rotatable digestion vessel into which the deceased body is loaded and/or the tissue digester system itself may include other agitator movements which are intended to facilitate or accelerate digestion. For example, the rotatable digestion vessel or the digestion chamber itself may oscillate, vibrate, rock, or apply ultrasonic vibration, without limitation. Other fluid circulation movements may be provided by the tissue digester system, for example, such as may be the result of a fluid circulating system.

In some embodiments of the various tissue digester devices, systems and methods described herein, digestive cycles may be carried out in lower temperature, non-pressurized systems, thereby to reduce the complexity of the system, to simplify maintenance and/or render the device, system or method safer to operate. With these embodiments of tissue digester devices, systems and methods, sufficient digestion of biological tissue may be achieved in a timeframe of two to three hours at a lower temperature, typically below boiling point, and in some embodiments, at approximately 96° C. or 205° F. In other embodiments, however, the digestive cycles may be carried out in higher temperature, pressurized systems, wherein the pressure allows operation at a higher temperature whilst preventing boiling of the digestive fluid.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A tissue digester device, which comprises:
a rotatable digestion vessel having a body which defines an internal chamber for receiving tissue;
a plurality of perforations through the body of the rotatable digestion vessel; and
a trough formed on the body of the rotatable digestion vessel, the trough extending into the internal chamber and configured to receive a caustic substance when the rotatable digestion vessel is stationary.

2. The tissue digester device of claim 1, wherein the trough is in the form of a v-shaped notch.

3. The tissue digester device of either one of claim 1 or claim 2, wherein the trough extends into the internal chamber to form an agitator within the internal chamber.

4. The tissue digester device of any one of claims 1 to 3, wherein the trough extends along a length of the body.

5. The tissue digester device of any one of claims 1 to 4, wherein at least a portion of the trough is impervious to the caustic substance.

6. The tissue digester device of any one of claims 1 to 5, wherein the rotatable digestion vessel comprises a lid adapted to provide access to the internal chamber, and wherein the trough is formed within the lid.

7. The tissue digester device of any one of claims 1 to 6, wherein the trough comprises at least one wall, and wherein the at least one wall comprises a lid which provides access to a secondary digestion vessel extending into the internal chamber between the at least one wall and the body.

8. The tissue digester device of any one of claims 1 to 7, shaped and dimensioned to be receivable into a digestion chamber such that the caustic substance is insertable into the trough without directly striking fluid held in the digestion chamber.

9. The tissue digester device of claim 8, wherein the digestion chamber comprises a rotation mechanism, and wherein the tissue digester device is connectable to the rotation mechanism.

10. The tissue digester device of either one of claim 8 or claim 9, wherein rotational movement of the rotatable digestion vessel mixes the caustic substance with fluid in the digestion chamber to form or supplement a digestive fluid.

11. The tissue digester device of any one of claims 1 to 10, wherein the tissue digester device is manufactured of steel.

12. A method of introducing a caustic substance to a digestion chamber, which comprises:
providing the digestion chamber having a rotatable digestion vessel arranged therein, the rotatable digestion vessel having a plurality of perforations and a trough;
arranging the rotatable digestion vessel within the digestion chamber such that the trough faces upwards;
inserting the caustic substance into the trough whilst stationary; and
rotating the rotatable digestion vessel such that the caustic substance egresses the trough into the digestion chamber.

13. The method of claim 12, comprising inserting fluid in the digestion chamber.

14. The method of claim 13, wherein the rotating of the rotatable digestion vessel mixes the caustic substance with the fluid to form or supplement a digestive fluid.

15. The method of any one of claims 12 to 14, comprising preloading the rotatable digestion vessel with biological tissue.

16. The method of any one of claims 12 to 15, wherein the rotatable digestion vessel comprises a secondary digestion vessel, and wherein the method comprises preloading the secondary digestion vessel with biological tissue.

17. The method of either one of claim 13 or claim 14, wherein the fluid comprises water, and wherein the caustic substance comprises any one or both of: potassium hydroxide (KOH) and sodium hydroxide (NaOH).

18. The method of any one of claims 12 to 17, wherein the rotatable digestion vessel comprises a tissue digester device as claimed in any one of claims 1 to 11.

19. A tissue digester system, which comprises:
a digestion chamber configured to contain a digestive fluid;
a rotatable digestion vessel arranged within the digestion chamber, having a substantially perforated body which defines an internal chamber adapted to receive a large tissue body for digestion; and
at least one secondary digestion vessel disposable within the internal chamber of the rotatable digestion vessel, having a substantially perforated body which defines a secondary internal chamber adapted to receive a smaller tissue body for digestion, in use the at least one secondary digestion vessel moves in conjunction with the rotatable digestion vessel such that the large tissue body and the smaller tissue body are simultaneously at least partially digested through contact with the digestive fluid.

20. The tissue digester system of claim 19, wherein the at least one secondary digestion vessel is removably attachable to an inner surface of the rotatable digestion vessel such that it protrudes into the internal chamber.

21. The tissue digester system of claim 20, wherein the at least one secondary digestion vessel is removably attachable to the inner surface with one or more fasteners.

22. The tissue digester system of claim 19, wherein the rotatable digestion vessel comprises at least one wall, and wherein the at least one secondary digestion vessel is formed by the at least one wall protruding into the internal chamber.

23. The tissue digester system of claim 19, wherein the at least one secondary digestion vessel is freely disposed within the internal chamber of the rotatable digestion vessel, the at least one secondary digestion vessel freely tumbling upon rotation of the rotatable digestion vessel.

24. The tissue digester system of claim 23, wherein the at least one secondary digestion vessel comprises a floatation pocket which provides floatation to the at least one secondary digestion vessel when in the digestive fluid.

25. The tissue digester system of any one of claims 19 to 24, wherein the at least one secondary digestion vessel comprises a lid through which access to the secondary internal chamber is provided.

26. The tissue digester system of claim 25, wherein the lid is any one of: a hinged lid, or a sliding lid.

27. The tissue digester system of any one of claims 19 to 26, wherein the at least one secondary digestion vessel is operable as a baffle or an agitator within the internal chamber of the rotatable digestion vessel.

28. The tissue digester system of any one of claims 19 to 27, wherein the at least one secondary digestion vessel is any one of: triangular, circular, square or polygonal, in cross-section.

29. The tissue digester system of any one of claims 19 to 28, wherein the rotatable digestion vessel comprises a trough, and wherein the at least one secondary digestion vessel is positioned along a length of an interior wall of the trough.

30. The tissue digester system of any one of claims 19 to 29, comprising at least one digestion cannister shaped and dimensioned to receive the smaller tissue body, the at least one digestion cannister receivable into the at least one secondary digestion vessel.

31. The tissue digester system of claim 30, wherein the at least one digestion cannister comprises a fine mesh.

32. The tissue digester system of any one of claims 19 to 31, wherein the rotatable digestion vessel comprises a tissue digester device as claimed in any one of claims 1 to 11.

33. A method of digesting small tissue bodies, which comprises:
providing a tissue digester system comprising
a digestion chamber configured to contain a digestive fluid;
a rotatable digestion vessel arranged within the digestion chamber, having a substantially perforated body which defines an internal chamber; and
at least one secondary digestion vessel disposed within the internal chamber of the rotatable digestion vessel, having a substantially perforated body which defines a secondary internal chamber;
loading a small tissue body to be digested into the secondary internal chamber of the at least one secondary digestion vessel;
adding digestive fluid to the digestion chamber; and
rotating the rotatable digestion vessel for a predetermined period to digest the small tissue body through exposure to the digestive fluid in a digestion cycle.

34. The method of claim 33, wherein the tissue digester system comprises at least one digestion cannister receivable into the secondary internal chamber of the at least one secondary digestion vessel.

35. The method of claim 34, wherein the loading of the small tissue body comprises loading the small tissue body into the at least one digestion cannister, and inserting the at least one digestion cannister into the secondary internal chamber of the at least one secondary digestion vessel.

36. The method of claim 35, comprising pausing the digestion cycle of the tissue digester system to remove the at least one digestion cannister from the at least one secondary digestion vessel upon sufficient digestion of the small tissue body.

37. The method of any one of claims 33 to 36, comprising loading a large tissue body into the internal chamber of the rotatable digestion vessel to be simultaneously digested with the small tissue body.

38. The method of any one of claims 33 to 37, wherein the adding of the digestive fluid comprises separately adding to the digestion chamber water and a caustic substance.

39. The method of any one of claims 33 to 38, wherein the small tissue body comprises a carcass under one kilogram in body weight.

40. The method of any one of claims 33 to 39, wherein the tissue digester system comprises a tissue digester system as claimed in any one of claims 19 to 32.

41. A head encasing device which is operable to mechanically facilitate digestion of brain tissue of a deceased body, which comprises:
a body which is shaped and dimensioned to receive and secure therein a head of the deceased body;
an aperture formed in the body which is shaped and dimensioned to surround a neck of the deceased body; and
an internal projection extending from an internal surface of the body proximate the aperture to engage a craniocervical region of the head, in use to pierce the craniocervical region such that digestive fluid can enter a cranium of the head to digest the brain tissue.

42. The head encasing device of claim 41, wherein the internal projection is arranged to pierce the craniocervical region in use such that the digestive fluid can enter a foramen magnum of the head.

43. The head encasing device of either one of claim 41 or claim 42, comprising an opposing internal projection arranged substantially opposite the internal projection, the internal projection and the opposing internal projection forming a pair of internal projections which engage the craniocervical region at an anterior position and a posterior position, respectively.

44. The head encasing device of claim 43, wherein the pair of internal projections comprise a pair of spikes.

45. The head encasing device of any one of claims 41 to 44, comprising one or more agitator projections extending from the internal surface of the body.

46. The head encasing device of claim 45, wherein the one or more agitator projections are arranged on the internal surface to engage temple regions of the head.

47. The head encasing device of either one of claim 45 or claim 46, wherein the one or more agitator projections are configured to fracture one or both pterions of the cranium on lateral sides of the head.

48. The head encasing device of any one of claims 41 to 47, wherein the body is at least partially perforated such that it is permeable to the digestive fluid.

49. The head encasing device of any one of claims 41 to 48, wherein the body comprises a two-part body.

50. The head encasing device of claim 49, wherein the two-part body comprises a hinged connection.

51. The head encasing device of any one of claims 41 to 50, wherein the body comprises a releasable fastener for securing the body in a closed configuration around the head and the neck.

52. The head encasing device of any one of claims 41 to 51, wherein the head is a human head, and wherein the body is substantially ovoid-shaped.

53. The head encasing device of any one of claims 41 to 52, which is manufactured of one or more non-digestible materials such that the head encasing device is reusable for a plurality of digestive cycles.

54. The head encasing device of any one of claims 41 to 53, wherein any one or both of the body and the internal projection is manufactured of steel.

55. A method of facilitating digestion of brain tissue in a head of a deceased body, which comprises:
loading the deceased body into a rotatable digestion vessel of a tissue digester system;
fitting a head encasing device to the head of the deceased body such that internal projections disposed within the head encasing device engage a craniocervical region of the deceased body;
inserting digestive fluid into the tissue digester system; and
rotating the rotatable digestion vessel such that the rotation thereof causes the head encasing device to impact an internal surface of the rotatable digestion vessel, urging the internal projections to pierce the craniocervical region to facilitate entry of the digestive fluid into a cranium of the head for digestion of the brain tissue.

56. The method of claim 55, wherein the piercing of the craniocervical region to facilitate contact between the digestive fluid and the brain tissue reaches a foremen magnum of the cranium.

57. The method of either one of claim 55 or claim 56, wherein the head encasing device comprises one or more agitator projections arranged on an internal surface of the head encasing device to engage temple regions of the head, and wherein impact of the head encasing device with the internal surface of the rotatable digestion vessel urges the one or more agitator projections to fracture one or both pterions of the cranium.

58. The method of any one of claims 55 to 57, wherein impact of the head encasing device with the internal surface assists in detachment of the head from the deceased body.

59. The method of any one of claims 55 to 58, wherein the head encasing device comprises a hinged two-part body, and wherein the fitting of the head encasing device to the head comprises opening the hinged two-part body, inserting the head in one part of the hinged two-part body, and closing another part of the hinged two-part body such that the hinged two-part body is secured to the head.

60. The method of any one of claims 55 to 59, wherein the head encasing device is a head cage device as claimed in any one of claims 41 to 54.
